SEQUENCE LISTING (2 sequences)

SEQ ID NO: 1 - DNA, 3215 bp, Homo sapiens
  misc_feature: alpha-synuclein  accession number NM_000345.3
aggagaaggagaaggaggaggactaggaggaggaggacggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 2 - protein, 140 aa, Homo sapiens
  MISC_FEATURE: alpha-synuclein  accession number NP_000336.1
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

(19) United States
(12) Patent Application Publication
(10) Pub. No.: US 2021/0041461 A1
(43) Pub. Date: Feb. 11, 2021

(54) DETECTION OF PHOSPHO-SERINE 129 ALPHA-SYNUCLEIN IN BLOOD CELLS AS A BIOMARKER FOR SYNUCLEINOPATHIES

(71) Applicant: YISSUM RESEARCH DEVELOPMEN COMPANY OF THE HEBREW UNIVERSITY OF JERUSALEM LTD, Jerusalem (IL)

(72) Inventor: Ronit SHARON, Mevasseret Zion (IL)

(21) Appl. No.: 16/979,822

(22) PCT Filed: Mar. 14, 2019
(86) PCT No.: PCT/IL2019/050289

§ 371 Date: Sep. 10, 2020

Related U.S. Application Data
(60) Provisional application No. 62/642,786, filed on Mar. 14, 2018.

(51) Int. Cl. G01N 33/68

(52) U.S. Cl. CPC G01N 33/6896; G01N 2800/52; G01N 2800/2835; G01N 2440/14

(57) ABSTRACT

The present invention relates to diagnostic methods and kits for the detection and/or diagnosis of at least one synucleinopathy in a subject. More particularly, the invention provides the use of a-Syn and its post translational modifications, specifically, serine 129 phosphorylated a-Syn, as diagnostic markers for Parkinson's disease specifically, for the diagnosis of PD with motor symptoms (PD-M).

### FIELD OF THE INVENTION

The present invention relates to diagnostic methods and kits. More particularly, the invention provides α-Synuclein protein (α-Syn) and its post translational modifications, specifically, serine 129 phosphorylated α-Syn, as diagnostic markers for Parkinson's disease, the synucleinopathy and cancer.

### BACKGROUND ART

References considered to be relevant as background to the presently disclosed subject matter are listed below:
[1] Mollenhauer B, et al. (2017) Perianalytical considerations. Mov Disord 2017, 32:1117-30.
[2] Matsuo Y, et al. (2010) PLoS One, 5:e10481.
[3] Locascio J J, et al. (2015) Brain; 138(Pt 9):2659-71.
[4] Vicente Miranda H, et al. (2017) Scientific reports. 2017; 7(1):13713.
[5] Foulds P G, et al. (2012) Neurobiol Dis; 45(1):188-95.
[6] Foulds P G, et al. (2013) Scientific reports; 3:2540.
[7] Barbour R, et al. (2008) Neurodegener Dis; 5(2):55-9.
[8] Nakai M, et al. (2007) Biochem Biophys Res Commun; 358(1):104-10.
[9] Scherzer C R, et al. (2018) Proc Natl Acad Sci USA; 105(31):10907-12.
[10] Foulds P G, et al. (2011) Faseb j. 2011; 25(12):4127-37.
[11] Michell A W, et al. (2005) Neurosci Lett; 381(3):294-8.
[12] Iwatsubo T, et al. (2002) Nat Cell Biol 2002, 4:160-4.
[13] Chilcote T J, et al. (2006) J Biol Chem 2006, 281:29739-52.
[14] Outeiro T F, et al. (2014) Front Mol Neurosci, 7:42.
[15] Allsop D, et al. (2012) Exp. rev. of mol. diagnostics, 12:115-7.
[16] Sharon R. et al. (2016) Anal Bioanal Chem 2016, 408:7669-77.
[17] WO/2014/132249.
[18] Samuel F, et al. (2016) J Biol Chem. 2016; 291(9):4374-85.
[19] WO2007/089862.
[20] Halliday et al. (2011) Acta Neuropathol 121:695-704.

Acknowledgement of the above references herein is not to be inferred as meaning that these are in any way relevant to the patentability of the presently disclosed subject matter.

### BACKGROUND OF THE INVENTION

The complex etiology of Parkinson's disease (PD) is only poorly understood. A growing evidence now suggests that neurodegeneration in PD is not restricted to the dopaminergic neurons localized to the substantia nigra. Rather, that PD is a systemic disease, involving peripheral tissues and may be caused by oxidative, metabolic, inflammatory, or biochemical processes (Cantello R, et al. (2014) Parkinsonism Relat Disord; 20(12):1329-34). The pathological hallmark of PD is the occurrence of Lewy pathology in the central nervous system (CNS), of which α-Syn protein is a major constituent. Lewy pathology also occurs in the peripheral nervous system, in neurons of the gastrointestinal tract and in the appendix, supporting a propagative disease model, starting at peripheral tissues and propagating to the CNS (Del Tredici K, et al. (2012) Mov Disord; 27(5):597-607; Braak H. et al. (2013) Nat Rev Neurol; 9(1):13-24). The development of a non-invasive and reliable biomarker that reflects the pathogenic process is a highly desired objective in the diagnosis and research of PD. Among the different factors associated with the pathogenic process of PD, α-Syn protein and its post-translational modified forms are most prominent.

Altered levels of synuclein proteins have been associated with a pathogenic condition based on its levels in the CNS, CSF, saliva and plasma of patients with PD and the related synucleinopathy and also in various types of cancer [1]. For example, α-Syn expression is detected in melanoma tumors and nevi [2].

The accessibility of the blood makes it a favorable sampling bio-fluid that can assist the follow up and treatment of a patient during the course of the disease. α-Syn in blood has been tested as a biomarker for PD [3-6]. However, it is important to emphasize the biology of α-Syn in the blood and the potential relevance to the disease. A principal source for α-Syn detected in the blood is blood-cells expressed α-Syn, particularly of erythroid lineage [7-9]. In addition, low levels of a neuronal-expressed, prion-like secreted α-Syn, may be found in blood plasma [6, 10]. The relevance of neuronal-secreted α-Syn to the disease is therefore clear. It represents a form that is closely associated with the pathogenic spread of the disease. However, the relevance of blood-cells expressed α-Syn to the pathogenesis of the disease is not fully understood yet. Blood cells-expressed α-Syn is mostly contributed by red blood cells [7]. Platelets and blood mononuclear cells also express α-Syn, however, at lower levels [7, 11]. α-Syn is subjected to several post-translational modifications, one such modification is phosphorylation at Serine129 (PSer129 α-Syn) [12]. Whereas only ˜5% of the soluble, monomeric α-Syn appears phosphorylated under physiological conditions in vivo, approximately 90% is phosphorylated in Lewy Bodies, in brains with PD [12-14].

Secreted PSer 129 α-Syn was detected in blood plasma [6, 10, 15]. However, a major obstacle in measuring plasma levels of PSer 129 α-Syn is the origin of this α-Syn form, which is critically affected by hemolysis, inevitably occurring during the process of blood sample collection. Importantly, the occurrence of PSer129 α-Syn in blood cells was not reported before. On the contrary, it was reported that the detection of PSer129 α-Syn in blood cells cannot be achieved [4].

A Lipid-ELISA method recently developed by the inventors, enables efficient capture of α-Syn from a test sample by immobilized lipids, followed by detection of α-Syn using antibodies. [16 and 17].

WO2007/089862 relates to an invention providing agents for treatment of diseases associated with Lewy Body diseases (LBD), specifically including inhibitors of kinases acting on α-synuclein such as PLK2 and GRK6 kinases [19].

Halliday et al. detected changes in the solubility and phosphorylation of α-synuclein in brain samples, over the course of Parkinson [20].

There is therefore a clear need for sensitive assays for detection of synucleinopathies and related conditions.

### SUMMARY OF THE INVENTION

A first aspect of the invention relates to a method for the detection and/or diagnosis of at least one synucleinopathy in a subject. In some embodiments, the method of the invention may comprise the steps of:

In a first step (a), determining the amount of α-synuclein (α-Syn) phosphorylated at Serine 129 (PSer129 α-Syn) in at least one biological sample of said subject, specifically, whole blood sample, or in at least one aliquot thereof; to obtain a PSer129 α-Syn value of the sample.

The next step (b), involves determining if the value obtained in step (a), is any one of positive or negative with respect to a predetermined standard PSer129 α-Syn value or to a PSer129 α-Syn value in at least one control sample. It should be noted that in some embodiments, a positive value of said PSer129 α-Syn in the sample, indicates that the tested subject suffers from at least one synucleinopathy.

A further aspect of the invention relates to a kit comprising:

(a) immobilized lipids and/or at least one immobilized hydrophobic agent; and (b) at least one agent that specifically recognizes and binds PSer129 α-Syn. In some embodiments, the kit of the invention may optionally further comprise at least one of:
(c) at least one agent that specifically recognizes and binds α-Syn; (d) Proteinase K;
(e) means for determining iron levels in a sample; (f) pre-determined calibration curve providing standard; (g) at least one control sample; and (h) at least one means for depleting hemoglobin from a whole blood sample.

A further aspect of the invention relates to a method for the assay of PSer129 α-Syn in a whole blood sample. More specifically, the method of the invention may comprise the steps of:

First in step (a), contacting said whole blood sample or at least one aliquot thereof with immobilized lipids and/or at least one immobilized hydrophobic agent, under conditions enabling binding of the synucleins to the lipids and/or hydrophobic agent.

In the next step (b), detecting the lipid-bound PSer129 α-Syn by at least one agent that specifically recognizes and binds said PSer129 α-Syn.

These and other aspects of the invention will become apparent as the description proceeds.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1F: α-Syn levels in samples of whole blood cells determined by Lipid-ELISA

FIG. 1A: Graph showing mean±SD of total α-Syn detected through binding to a mixture of PI:PS:PE:GM1 (1:1:1:1) that were immobilized to the ELISA plate using methanol as a solvent. HC, healthy controls; PD-M, PD with motor symptoms; PD-D, PD with cognitive symptoms

FIG. 1B: Graph showing correlation of the total α-Syn levels with total UPDRS (I+II+III) scores, using linear regression analysis with Pearson's correlation.

FIG. 1C: Graph showing mean±SD of PK^{res} α-Syn detected through binding to a mixture of PI:PS:PE:GM1 (1:1:1:1) that were immobilized to the ELISA plate using methanol as a solvent. HC, healthy controls; PD-M, PD with motor symptoms; PD-D, PD with cognitive symptoms

FIG. 1D: Graph showing correlation of the PK^{res} α-Syn levels with total UPDRS (I+II+III) scores, using linear regression analysis with Pearson's correlation.

FIG. 1E: Graph showing mean±SD of PSer129 α-Syn detected through binding to a mixture of PI:PS:PE:GM1 (1:1:1:1) that were immobilized to the ELISA plate using methanol as a solvent.

FIG. 1F: Graph showing correlation of the PSer129 α-Syn levels with total UPDRS (I+II+III) scores, using linear regression analysis with Pearson's correlation.

HC, healthy controls; PD-M, PD with motor symptoms; PD-D, PD with cognitive symptoms. *, P<0.05; **, P<0.01 (Kruskal Wallis test).

FIG. 2A-2C: A composite biomarker differentiates PD with motor symptoms (PD-M) and healthy controls (HC)

FIG. 2A: Graph showing the distribution of the composite biomarker, that is calculated by logistic regression of the concentrations of total α-Syn, PK^{res} α-Syn and PSer129 α-Syn, and iron levels in HC, PD-M and PD-D groups.

FIG. 2B: Graph showing the correlation between the composite biomarker in PD-M with UPDRS (I+II+III).

FIG. 2C: ROC curve showing the strength of the composite biomarker in differentiating the PD-M and HC groups.

FIG. 3: Graph showing detection assay of purified α-synuclein and purified PSer129 α-Syn with or without CHAPS in wash solutions Immobilized lipids were PI:PS:PE:GM-1 dissolved in cyclohexene. Detection was performed using anti PSer129 α-Syn antibody ((WAKO, Clone pSyn#64)).

### DETAILED DESCRIPTION OF THE INVENTION

The present invention shows, for the first time, the occurrence of PSer129 α-Syn in blood cells, and its detection by a lipid ELISA assay. PSer129 α-Syn is detected due to its expression in blood cells contrary to PSer129 α-Syn originated from affected nerve cells as detected in CNS or plasma, due to its biochemical property to bind membrane lipids.

Moreover, the present invention demonstrates for the first time, the usefulness of blood cells expressed α-Syn, as a biomarker for PD. The levels of total α-Syn, PK^{res} α-Syn and PSer129 α-Syn were determined by a lipid-ELISA assay and found to significantly differ between a group of healthy individuals and a group of individuals affected with PD, presenting motor symptoms (PD-M) without dementia. The concentrations of the three α-Syn forms and iron were used to develop a predictive model capable of differentiating the PD-M and healthy control (HC) groups. Cross validation of the model provided an AUC (95% CI) of 0.85 (0.77-0.94) with a high specificity values (0.91). In addition, the results of the resent invention clearly demonstrate differences in these variables between PD sub groups, PD-M and PD-D. The composite α-Syn biomarker of the invention, measured in blood cells, meets the definition of a useful biomarker for the diagnosis of synucleinopathies, and specifically, PD.

Thus, a first aspect of the invention relates to a method for the detection and/or diagnosis of at least one synucleinopathy in a subject. In some embodiments, the method of the invention may comprise the steps of:

In a first step (a), determining the amount of α-synuclein (α-Syn) phosphorylated at Serine 129 (PSer129 α-Syn) in at least one biological sample of said subject, specifically, whole blood sample, or in at least one aliquot thereof; to obtain a PSer129 α-Syn value of the sample.

The next step (b), involves determining if the value obtained in step (a) is any one of positive or negative with respect to a predetermined standard PSer129 α-Syn value or to a PSer129 α-Syn value in at least one control sample. It should be noted that in some embodiments, a positive value of the PSer129 α-Syn in the sample, indicates that the tested subject suffers from at least one synucleinopathy.

The methods of the present invention therefore use the levels of synucleins, specifically, a post translationally modified alpha synuclein, and more particularly, PSer129 α-Syn, as a diagnostic and/or prognostic biomarker for synucleinopaties, and related conditions, disorders or symptoms. Synucleins are a family of soluble proteins common to vertebrates, primarily expressed in neural tissue and in certain tumors. The synuclein family comprises three types of proteins: alpha-synuclein, beta-synuclein, and gamma-synuclein. Interest in the synuclein family began when alpha-synuclein was found to be mutated in several families with autosomal dominant Parkinson's disease. All synucleins have in common a highly conserved alpha-helical lipid-binding motif with similarity to the class-A2 lipid-binding domains of the exchangeable apolipoproteins.

In some embodiments, the methods of the invention are based particularly on assaying a post translationally modified α-synuclein (α-Syn) in a biological sample, specifically, whole blood sample. Alpha-synuclein (α-Syn), is a protein abundant in the brain while smaller amounts are found in the heart, muscles, and other tissues. In the brain, alpha-synuclein is found mainly at the tips of nerve cells (neurons) in specialized structures called presynaptic terminals Within these structures, alpha-synuclein is known to directly bind to lipid membranes, associating with the negatively charged surfaces of phospholipids.

Although the function of alpha-synuclein is not well understood, studies suggest that it plays a role in maintaining a supply of synaptic vesicles in presynaptic terminals by clustering synaptic vesicles. It may also help regulate the release of dopamine

The human alpha-synuclein protein is made of 140 amino acids and is encoded by the SNCA gene. In some embodiments, the methods of the invention are specifically applicable for human α-Syn. More specifically, the human alpha-synuclein protein used by the methods and kits of the invention as a biomarker, is encoded by the nucleic acid sequence as denoted by NM_000345.3. In yet some further specific embodiments, such alpha-synuclein protein is encoded by a nucleic acid sequence comprising the nucleic sequence as denoted by SEQ ID NO: 1. In yet some further embodiments, the human alpha-synuclein protein may comprise the amino acid sequence as denoted by NP_000336.1. In more specific embodiments, the human α-Syn of the invention may comprise the amino acid sequence as denoted by SEQ ID NO: 2.

As indicated above, the methods of the invention specifically involve determination of the amount or the levels of post translationally modified α-Syn, specifically, α-Syn that is phosphorylated at serine residue 129. In some embodiments, the serine residue 129, refers to the serine residue at position 129 of the human α-Syn as shown by the amino acid sequence denoted by SEQ ID NO. 2.

Still further, the terms “amount” or “level” of post translationally modified α-Syn, specifically, PSer129 α-Syn, are used interchangeably, and generally refer to a numerical representation of the amount (quantity) of an amino acid product or polypeptide or protein in a biological sample (mg, μg, pg etc.).

The methods of the invention, as well as the kits disclosed herein after, refer to the level or amount of the biomarker protein/s (e.g., PSer129 α-Syn and optionally, proteinase K resistant α-Syn and total α-Syn) in the sample. It should be understood that the level of PSer129 α-Syn reflects the level of expression of α-Syn in the subject, the level of the post translational modifications, specifically, the phosphorylation in the subject, and may also reflect the stability of the protein, specifically, in its translationally modified form.

The amount or level of post translationally modified α-Synucleins, specifically, the PSer129 α-Syn of the invention, is determined to obtain a PSer129 α-Syn value of the sample. The term “PSer129 α-Syn value” refers to the result of a calculation, that uses as an input the amount of PSer129 α-Syn obtained experimentally, for example by measuring the absorbance of the protein sample in 280 mm (Optical density (O.D.)). In some embodiments, this amount is calculated and determined with respect to a calibration curve of known amounts of PSer129 α-Syn. In some specific embodiments, P-Ser129 α-Syn levels in blood cells are determined according to a standard curve of purified recombinant P-Ser129 α-Syn protein (purchased from MJFF resources). It should be appreciated that in some optional embodiments, determination of the value may further involves normalizing the measured “amount of the PSer129 α-Syn” by at least one normalization step as detailed herein, where the resulting calculated value termed herein “PSer129 α-Syn value” is obtained. More specifically, as used herein, “normalized values” in some embodiments, are the quotient of raw values of post translationally modified α-Syn proteins, specifically, PSer129 α-Syn, divided by the value of a control reference protein from the same sample, or in some embodiments, the total α-Syn value determined for the sample. Thus, in some embodiments, the PSer129 α-Syn amount may be normalized to 1 mg of total α-Syn. This normalized value may then be compared with normalized cutoff values, i.e., cutoff values calculated from normalized values. In certain embodiments, the control reference protein may be a protein that maintains stable in all samples analyzed.

Normalized PSer129 α-Syn values that are higher (positive) or lower (negative) in comparison with a corresponding predetermined standard value or a cut-off value in a control sample predict to which population of subjects, either healthy or diseased, the tested sample belongs. In some embodiments, the values may even reflect the disease stage, or the metastatic status of the subject, in case a synucleopathy associated condition such as cancer is diagnosed. It should be appreciated that an important step in the methods of the inventions is determining whether the protein value of the post translationally modified α-Syn protein, is changed or different when compared to a pre-determined standard value, a control sample or a predetermined cut off, or alternatively, is within the range of amount of such cutoff.

Thus, in yet more specific embodiments, the second step (b) of the method of the invention involves comparing the values determined for the tested sample with predetermined standard values or cutoff values, or alternatively, with values determined for at least one control sample. As used herein the term “comparing” denotes any examination of the level and/or values obtained in the samples of the invention as detailed throughout in order to discover similarities or differences between at least two different samples. It should be noted that in some embodiments, comparing according to the present invention encompasses the possibility to use a computer based approach. This comparison enables determining if the tested subject is “positive” or “negative”, thereby determining if the tested subject is affected with any synucleopathy or any related condition.

Still further, when a quantitative determination is being performed, the results obtained from the assay of the present invention is compared with results obtained with standardized amounts of pure P-Ser129 α-Syn and/or with results obtained from populations of healthy subjects and/or groups of patients having the relevant disease. In some particular embodiments, the results obtained with a patient's sample are compared with average values obtained from a standard set of results previously obtained from a cohort of patients. In yet some other embodiments (for example, when the assay is being used for diagnostic purposes), the results obtained with the patient's sample will be compared with a cut off value previously obtained from a standard set of results. A positive diagnosis (i.e. presence of the disease) is reached when the results obtained with the patient's sample is significantly different from the pre-determined cutoff value. A negative diagnosis (i.e., no indication for the disease) is reached when the results obtained in a patient's sample is not different from the reference values. In most such cases, a positive diagnosis is obtained when the PSer129 α-Syn concentration measured in the patient's sample is significantly higher than the reference value. However, in certain instances, a significant reduction in synuclein concentration (when compared with the control reference value) will be used as the indicator of the presence of the disease. (That is, the PSer129 α-Syn value changes in accordance with disease progression). It is higher in early stages of the disease (e.g., in stages involving motor symptoms) and lower in advanced stages of the disease (e.g., in stages involving dementia).

For detection purposes, the method of the invention may be used for detection of a synucleinopathy or cancer in an individual from which the sample was obtained, wherein the level of immobilized PSer129 α-Syn (immobilized with the lipids) is compared to one or more reference values obtained from groups of healthy individuals and/or patients diagnosed with the relevant synucleinopathy or cancer. A level in the tested sample, which is significantly different from the reference value(s), indicates the presence of synucleinopathy or cancer in the individual from which said sample was obtained.

Still further, in some particular and non-limiting embodiments, for calculating and determining pre-determined standard values and/or cutoff values used for the methods and kits of the invention, commercially available samples may be used. Non-limiting example for such samples are BioFIND samples. BioFIND (Fox Investigation for New Discovery of Biomarkers in Parkinson's Disease) is a cross-sectional, multicenter biomarker study that established a repository of clinical data, blood, DNA, RNA, CSF, saliva, and urine samples from 118 moderate to advanced PD and 88 healthy control subjects. Inclusion criteria were designed to maximize diagnostic specificity by selecting participants with clinically typical PD symptoms, and clinical data and biospecimen collection utilized standardized procedures to minimize variability across sites.

BioFIND carefully standardized study procedures to minimize pre-analytical variability associated with sample processing and utilized the same procedures, wherever possible, as those used in PPMI, thereby enhancing further cross-study comparisons.

Detailed and standardized biospecimen collection, processing, and shipping (e.g., volume, aliquoting methods, centrifuge speeds and times, and so on) ensured the highest quality and uniformity of preanalytical variables in the sample collection. Additional details are available in the BioFIND Laboratory Manual (Supporting Information), and laboratory case report form data are available through the BioFIND database repository. It should be further appreciated that any standard value or cutoff calculated from any other known population of diagnosed patients or healthy subjects is also suitable for the methods and kits of the invention.

In some embodiments, the PSer129 α-Syn value of the sample is obtained by a method comprising: first is step (a), contacting the whole blood sample or at least one aliquot thereof with immobilized lipids, and/or at least one immobilized hydrophobic agent, under conditions enabling binding of the synuclein to the lipids and/or hydrophobic agent, specifically, immobilized lipids or hydrophobic agent; and in step (b), detecting the lipid-bound and/or hydrophobic agent-bound PSer129 α-Syn by at least one agent that specifically recognizes and binds the lipid-bound or hydrophobic agent-bound PSer129 α-Syn.

Thus, according to some embodiments, the invention provides methods for the detection and/or diagnosis of at least one synucleinopathy in a subject, comprising the steps of:

First (a), determining the amount of PSer129 α-Syn in at least one whole blood sample of the subject by: (i) contacting the whole blood sample or at least one aliquot thereof with immobilized lipids and/or immobilized hydrophobic agent/s, under conditions enabling binding of the synucleins to the lipids and/or at least one hydrophobic agent; and (ii), detecting the lipid-bound and/or hydrophobic agent-bound PSer129 α-Syn by at least one agent that specifically recognizes and binds the lipid-bound and/or the hydrophobic agent-bound, PSer129 α-Syn; to obtain a PSer129 α-Syn value of the sample.

The next step (b), involves determining if the value obtained in step (a) is any one of positive or negative with respect to a predetermined standard PSer129 α-Syn value or to a PSer129 α-Syn value in at least one control sample.

It should be noted that a positive value of said PSer129 α-Syn in the tested sample, indicates that the tested subject suffers from at least one synucleinopathy, or any symptoms or conditions associated therewith (e.g., cancer).

In some embodiments, the biological sample is a whole blood sample that comprises blood cells. The term, “blood cell” or “hematocyte” refers to a cell produced through hematopoiesis and is found in blood. In mammals, these cells fall into three general categories: red blood cells (erythrocytes), white blood cells (leukocytes), and platelets (thrombocytes). Together, these three kinds of blood cells add up to a total 45 percent of the blood tissue by volume, with the remaining 55 percent of the volume composed of plasma, the liquid component of blood. Peripheral blood mononuclear cells (PBMCs) comprise of any blood cell having a round nucleus (as opposed to a lobed nucleus), a lymphocyte or a monocyte.

The blood cells can be extracted in some embodiments, from whole blood using ficoll, a hydrophilic polysaccharide that separates layers of blood, and gradient centrifugation, which separates the blood into a top layer of plasma, followed by a layer of PBMCs and a bottom fraction of polymorphonuclear cells (such as neutrophils and eosinophils) and erythrocytes. The polymorphonuclear cells can be further isolated by lysing the red blood cells. Exemplary blood cells include erythrocytes, megakaryocytes, monocytes, and granulocytes. Human peripheral blood mononuclear cells (hPBMCs) are human blood cells (e.g., a lymphocyte or a monocyte) with a round nucleus. In addition, blood cells can be separated from plasma by standard (non-gradient) centrifugation or filtration. It should be understood that the whole blood sample used by the methods and kits of the invention may comprise any fraction or preparation of blood (e.g., protein preparation), provided that the sample is not a plasma sample. Thus, in some embodiments, a sample may be obtained from whole blood, and may comprise erythrocytes, platelets, white blood cells or any other type of blood cells, either intact or lysed, as long as the sample is not plasma. In some further embodiments, the blood sample is a sample of erythrocytes, or a mixture of erythrocytes and platelets. In some further embodiments, the sample is a sample of erythroblasts or polyreticulocytes. In further embodiments, the sample is a sample consisting of blood cell pellets or any lysates or preparations thereof (e.g., protein preparations), specifically, blood cells lysate.

In accordance with some embodiments of the invention, the sample may be filtered through size-limiting filters or similar, to eliminate the occurrence of undesired protein residents in the test sample, such as hemoglobin. Still further, in accordance with a specific embodiment of the invention, the sample may be treated with specific agents, to remove undesired proteins from the test sample.

In yet some further embodiments, the blood sample is a hemoglobin depleted sample. In some embodiments, for depletion of hemoglobin from a blood sample, any affinity method may be used. In yet some further embodiments, the commercially available HemoVoid™ may be used for such purpose. HemoVoid™, removes hemoglobin from erythrocyte lysate samples allowing for subsequent detection, identification and quantification of depleted hemoglobin samples. This step is used for overcoming the interference by high-abundance proteins obscuring less-abundant proteins. HemoVoid derives from a silica-based library of individual mixed-mode ligand combinations. The library is designed to facilitate weak binding of proteins, allowing for rapid elution from the matrix without any foreknowledge of the variety of proteins contained in the starting sample. Alternative methods for hemoglobin removal may include affinity columns (antibodies for hemoglobin) and Haptoglobin-based removal, products.

In yet some further embodiments of the invention, prior to contact with the immobilized membrane-forming lipids and/or immobilized hydrophobic agents, the sample may optionally be pretreated at a temperature of 30-95° C., preferably 95° C., for a period of 10 minutes to 24 hours. It should be understood that although specifically applicable for whole blood samples, the method of the invention may be performed efficiently using any other sample, with the proviso that the sample is not a serum sample. To name but few, biological samples applicable herein may include saliva, urine, tissue extracts, bone marrow, lymph fluid, blood cells, blood, sputum, faeces, semen, spinal fluid or CSF, the external secretions of the skin, respiratory, intestinal, and genitourinary tracts, tears, milk, any human organ or tissue, any sample obtained by lavage, plural effusion, sample of in vitro or ex vivo cell culture and cell culture constituents.

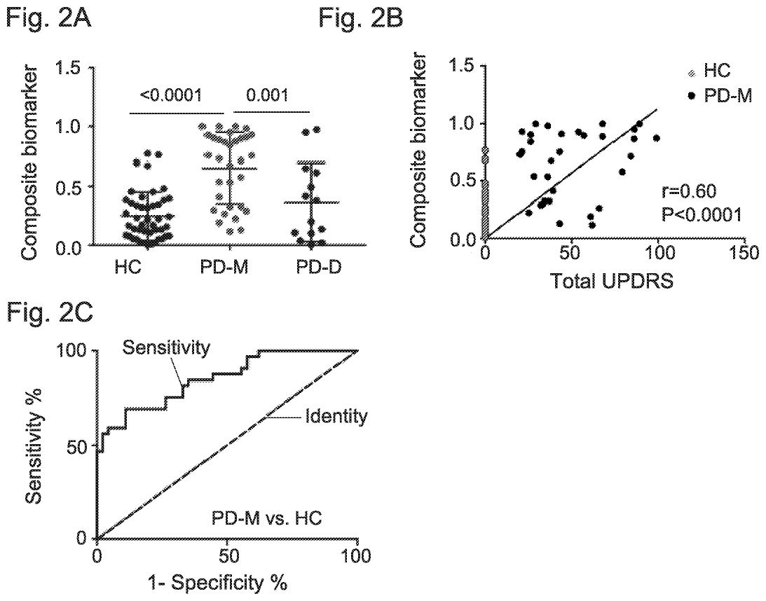

As shown by the following examples, specifically in FIG. 2, combination of several parameters improves the specificity and sensitivity of the diagnostic methods of the invention as discussed herein. Thus, in some embodiments, the method of the invention further comprises the steps of determining the value of at least one additional parameter in the tested sample or in at least one aliquot thereof. In some specific embodiments the method of the invention comprise the steps of:

First, in step (a), determining the amount of PSer129 α-Syn in at least one whole blood sample of the subject, or in at least one aliquot thereof, to obtain a PSer129 α-Syn value of the sample.

The next step (b), involves determining in at least one aliquot of the tested sample at least one of the following parameters:

(i) total α-Syn amount, to obtain an α-Syn value of the sample;
(ii) amount of proteinase K (PK)-resistant α-Syn, to obtain a PK resistant α-Syn value of the sample;
(iii) iron level, to obtain an iron value of the sample;
(iv) amount of oxidized α-Syn, to obtain an oxidized α-Syn value of the sample;
(v) amount of S-nitrosylated α-Syn, to obtain an α-Syn value of the sample;
(vi) amount of heat-resistant α-Syn, to obtain an α-Syn value of the sample;
(vii) hemoglobin level to obtain an α-Syn value of the sample; and
(viii) H-ferritin level, to obtain an α-Syn value of the sample.

The next step (c), involves calculating the weighed sum of the PSer129 α-Syn value as determined in step (a), and of at least one of the values of the at least one parameter as defined in step (b), specifically, the values of at least one of the parameters defined in (i) to (viii), to obtain a Sum value of the sample.

In the next step (d), determining if the Sum value obtained in step (c) is any one of positive or negative with respect to a predetermined standard Sum value or to a Sum value in at least one control sample.

In some embodiments, a positive Sum value indicates that the subject suffers from at least one synucleinopathy, or any conditions or symptoms associated therewith.

In some embodiments, the amount of oxidized α-Syn is determined as in step (a). The amount of S-nitrosylated α-Syn, is determined by an agent that recognizes S-nitrosylated α-Syn, for example, an antibody such as Syn303 antibody (Abcam). The amount of heat-resistant α-Syn is determined by preheating the sample, prior to contacting with the immobilized lipids, for about 10-30 minutes, at a temperature of 30° C.-95° C. Samples are then cooled down. The amount of heat resistant α-Syn is determined as in step (a).

The methods of the present invention enable the determination of levels of immobilized total α-Syn in combination with levels of immobilized modified alpha synuclein. That is, the levels of immobilized PSer129 α-Syn alone or levels of immobilized PK-α-Syn, or the ratio between these parameters. In yet some further embodiments, the ratio between immobilized and non-immobilized α-Syn, or non-immobilized PSer129 α-Syn. In yet some further embodiments, the ratio between the PSer129 α-Syn and non-immobilized PK-α-Syn. Still further, diagnosis may be based on either PSer129 α-Syn values, its combination with PK-α-Syn, total α-Syn, iron levels, or all.

In some specific embodiments, the method of the invention combines the following parameters for the diagnosis and detection of synucleinopathies in a tested subject. Thus, in some embodiments the methods of the invention may comprise the steps of:

In a first step (a), determining the amount of PSer129 α-Syn in at least one whole blood sample of the tested subject, or in at least one aliquot thereof, to obtain a PSer129 α-Syn value of the sample.

In the next step (b), determining in at least one aliquot of the tested sample the total α-Syn amount, to obtain an α-Syn value of the sample.

In step (c), determining in at least one aliquot of the tested sample the amount of proteinase K-resistant α-Syn, to obtain a PK resistant α-Syn value of the sample.

In step (d), determining in at least one aliquot of the tested sample the iron level, to obtain an iron value of the sample.

The next step (e), involves calculating the weighed sum of the measured parameters, specifically, the PSer129 α-Syn value as determined in step (a), the α-Syn value as determined in step (b), the PK resistant α-Syn value as determined in step (c), and the iron value as determined in step (d), to obtain a Sum value.

In the next step (f), determining if the Sum value obtained in step (e) is any one of positive or negative with respect to a predetermined standard Sum value, a cut off value, or a Sum value calculated for the same parameters in at least one control sample.

It should be noted that in some embodiments, a positive Sum value calculated in the sample, indicates that the tested subject suffers from at least one synucleinopathy, or any diseases, symptoms and conditions associated therewith.

As shown by the following examples, to distinguish and differentiate between the examined groups of subjects [e.g., healthy subjects, and different subgroups of Parkinson's disease patients (PD), specifically, with motor symptoms (PD-M), and with dementia (PD-D)], a diagnostic algorithm was developed using progressive regression tools. These tools evaluate the relative contribution of each of the examined and measured parameters (specifically, amount of protein, e.g., PSer129 α-Syn, total α-Syn, PK-resistant α-Syn and the amount of iron), to obtain the weighed values for each parameter. The weighed values were used therefore to calculate the weighed sum of the measured parameters using the following equation:

Z=a+(b×iron)+(c×total MeOH)+(d×PKres)+(e×PSer129)

Non-limiting embodiments for the weighed parameters is disclosed by Example 5.

This equation enables determination of the calculated Z value, that in some optional and non-limiting embodiments, is also referred to herein as the Sum value calculated for the sample by step (e) of the methods of the invention. The Z is used to calculate P(predict), a value used to determine the degree of discrimination between the test groups.

$$P ( predict ) = \frac{1}{1 + {e}^{- Z}}$$

In some embodiments, the Cut off is set at 0.5. Thus, where the calculated Sum value of the sample is below 0, the P will be determined as below the cutoff, specifically, below 0.5, and the sample is determined as “negative”, specifically, a subject that is not affected by a synucleopathy or any related condition. In yet some further embodiments, where the calculated Sum value of the sample is above 0, the P will be determined as above the cutoff, specifically, above 0.5, and the sample is determined as “positive”, specifically, a subject that is affected by a synucleopathy or any related condition.

As described hereinabove, the method of the invention refers to a predetermined cutoff value/s. It should be noted that a “cutoff value”, sometimes referred to simply as “cutoff” herein, is a value that meets the requirements for both high diagnostic sensitivity (true positive rate) and high diagnostic specificity (true negative rate).

It should be noted that the terms “sensitivity” and “specificity” are used herein with respect to the ability of the PSer129 α-Syn protein, to correctly classify a sample as belonging to a pre-established population associated with at least one synucleinopathy or cancer, or alternatively, to a pre-established population of healthy subjects or subjects that are not affected by at least one synucleinopathy or cancer.

“Sensitivity” indicates the performance of the post translationally modified α-Syn protein, specifically, PSer129 α-Syn of the invention, with respect to correctly classifying samples as belonging to pre-established populations that are likely to suffer from a disease or disorder or characterized at different stages of a disease.

“Specificity” indicates the performance of the post translationally modified α-Syn protein of the invention, specifically, PSer129 α-Syn with respect to correctly classifying and distinguishing between samples as belonging to pre-established populations of subjects suffering from the same disorder and populations of subjects that are either healthy or not affected by at least one synucleinopathy or cancer.

Simply put, “sensitivity” relates to the rate of identification of the patients (samples) as such out of a group of samples, whereas “specificity” relates to the rate of correct identification of synucleinopathy or cancer samples as such out of a group of samples. Cutoff values may be used as control sample/s or in addition to control sample/s, said cutoff values being the result of a statistical analysis of the post translationally modified α-Syn protein value/s differences in pre-established populations healthy or suffering from at least one synucleinopathy or cancer.

The diagnostic and prognostic methods of the invention involve the steps of determining if the value measured or calculated in the tested sample (e.g., the PSer129 α-Syn, or the Sum value calculated for several diagnostic parameters), is positive or negative with respect to a standard value (cutoff) predetermined in a control population or control sample/s. It should be therefore understood that in some embodiments, the control populations may be a population of healthy subjects, a population of subjects diagnosed with the same synocleinopathy or cancer or population of patients diagnosed with any other disorder. In yet some further embodiments, a population of subjects as used herein refer to at least two subjects, specifically, at least 2, 3, 4, 5, 6, 7, 8, 9, 20 or more subjects, 15, 20, 25, 30, 35, 40, 45, 50, 55, 60, 65, 70, 75, 80, 85, 90, 95, 100 or more subjects. In some embodiments, the subjects of these control populations are pre-diagnosed using any known diagnostic methods and tools. Of particular interest are any diagnostic tools and methods used for diagnosing patients suffering from at least one synucleinopathy. In non-limiting embodiments, such parameters may include at least one of: the mean change in the motor score (part III) of the unified Parkinson's disease rating scale (UPDRS score); Mean change in total UPDRS score (I-III); Hoehn and Yahr scale; Montreal Cognitive Assessment (MoCA) score; REM Sleep Behavior Disorder Screening Questionnaire (RBDSQ); Timed up-and-go test; Purdue pegboard; Neurotrax; The Patient Global Impression of Improvement (PGI-I); Parkinson's disease questionnaire (PDQ-39); Epworth Sleepiness Scale; Beck Depression Inventory; Frontal assessment battery (FAB); Addenbrooke's Cognitive Examination; Questionnaire for Impulsive-Compulsive Disorders in Parkinson's (QUIP-RS), Smell test; Substantia nigra (SN) ultra-sound hyperechogenicity (>0.2); Thinning of the retina measured by OCT; Lyso Gb1; Color discrimination test; Orthostatic hypotension, Genomics and transtriptomics tests for DNA mutations in genes associated with synucleinopathy or RNA transcript abundance, a proteomic tests showing changes in amount of α-Syn or its PTM, and even Microbiome tests may be also applicable.

More specifically, PSer129 α-Syn value or calculated Sum value that is determined by the method of the invention as “positive” when compared to a predetermined cutoff of population of patients suffering from synucleinopathy or cancer, or for at least one known patient suffering from synucleinopathy or cancer, may indicate that the examined subject belongs to a population suffering from synucleinopathy or cancer, in case that the expression value is either higher (positive) or fall within the range (the average values of the cutoff predetermined for patient population suffering from synucleinopathy or cancer). In other words, a positive value indicates that the subject is suffering from a synucleinopathy or cancer. More specifically, a positive value indicates that the subject suffers from PD, specifically, PD-M. In a similar manner, a subject exhibiting a PSer129 α-Syn value or calculated Sum value that is “negative” (that is lower) as compared to the cutoff patients, may be considered as belonging to population that is not suffering from synucleinopathy or cancer. In more specific embodiments, the value of such subject should fall within the range of the cutoff value predetermined for population that is not suffering from synucleinopathy or cancer. In other words, a negative Sum value indicates that the subject is not suffering from a synucleinopathy or cancer. More specifically, a negative value indicates that the subject is a healthy subject or at least, this subject is not suffering from PD, specifically, not suffering from PD-M. In some embodiments, “fall within the range” encompass values that differ from the cutoff value in about 1%, about 5%, about 10%, about 15%, about 20%, about 25%, about 30%, about 35%, about 40%, about 45%, about 50% or more.

The post translationally modified α-Syn values are selected along the ROC curve for optimal combination of diagnostic sensitivity and diagnostic specificity which are as close to 100 percent as possible, and the resulting values are used as the cutoff values that distinguish between subjects who are diagnosed with at least one synucleinopathy or cancer at a certain rate, and those who will not (with said given sensitivity and specificity) Similar analysis may be performed for example when diagnosis of synucleinopathy or cancer is being examined to distingue between healthy tissue and affected tissue. The ROC curve may evolve as more and more data and values are recorded and taken into consideration, modifying the optimal cutoff values of the weighed parameters and improving sensitivity and specificity. Thus, it should be appreciated that the provided cutoff values (e.g., 0.5), should be viewed as a starting point that may shift as more data allows more accurate cutoff value calculation. Although considered as initial cutoff values, the presently provided values already provide good sensitivity (e.g., 0.69) and specificity (e.g., 0.91), and are readily applicable in current clinical use, even in patients diagnosed with different synucleinopathy or cancer stages.

As noted above, the value obtained for a PSer129 α-Syn, or the Sum value of the weighed parameters (e.g., iron, total α-Syn and proteinase K resistant α-Syn) determined for the examined sample is compared with a predetermined cutoff or a control sample/s. More specifically, in certain embodiments, the value obtained for the examined sample is compared with a predetermined standard or cutoff value.

In further embodiments, the predetermined standard value, or cutoff value has been pre-determined and calculated for a population comprising at least one of healthy subjects, subjects suffering from any disorder, subjects suffering from different stages of any disorder, subjects that respond to treatment, non-responder subjects, subjects in remission and subjects in relapse.

Still further, in certain alternative embodiments where a control sample is being used (instead of, or in addition to, pre-determined cutoff values), the normalized values of the post translationally modified α-Syn proteins, specifically, PSer129 α-Syn used by the invention in the test sample are compared to the values in the control sample. In certain embodiments, such control sample may be obtained from at least one of a healthy subject, a subject suffering from a disorder at a specific stage, a subject suffering from a disorder at a different specific stage a subject that responds to treatment, a non-responder subject, a subject in remission and a subject in relapse.

It should be appreciated that “Standard” or a “predetermined standard” as used herein, denotes either a single standard value or a plurality of standards with which the level at the post translationally modified α-Syn proteins, specifically, Ser 129 phosphorylated α-Syn, and optionally, at least one of the other parameters assayed, 9Sum value) from the tested sample is compared. The standards may be provided, for example, in the form of discrete numeric values or is calorimetric in the form of a chart with different colors or shadings for different levels of amount of protein measured (or in some optional embodiments, iron levels); or they may be provided in the form of a comparative curve prepared on the basis of such standards (standard curve).

It should be noted that such signal-to-expression level data may be calculated and derived from a calibration curve.

Thus, in certain embodiments, the methods and kits of the invention may optionally further involve the use of a calibration curve created by detecting a signal for each one of increasing pre-determined concentrations of the post translationally modified α-Syn proteins, specifically, Ser 129 phosphorylated α-Syn, and optionally, at least one of the other parameters assayed. Obtaining such a calibration curve may be indicative to evaluate the range at which the levels correlate linearly with the concentrations of the post translationally modified α-Syn proteins. It should be noted in this connection that at times when no change in level of the post translationally modified α-Syn proteins is observed, the calibration curve should be evaluated in order to rule out the possibility that the measured level is not exhibiting a saturation type curve, namely a range at which increasing concentrations exhibit the same signal.

It must be appreciated that in certain embodiments such calibration curve as described above may be also part or component in any of the kits and methods provided by the invention as described herein after.

In some embodiments, the total α-Syn value of the sample is obtained by a method comprising: first (a), contacting the sample or at least one aliquot thereof with immobilized lipids under conditions enabling binding of the synucleins to the lipids and/or at least one hydrophobic agent; and (b), detecting the lipid-bound and/or hydrophobic agent-bound, α-Syn by at least one agent that specifically recognizes and binds the lipid-bound α-Syn, to obtain a total α-Syn value of the sample. In some specific embodiments, antibodies or aptamers specific for α-Syn are used to determine the amount of the total α-Syn in the sample.

In yet some further embodiments of the methods of the invention, a PK resistant α-Syn value of the sample is obtained by a method comprising: first (a), contacting said sample or at least one aliquot thereof with proteinase K; next, in step (b), contacting the proteinase K treated sample obtained in step (a) with immobilized lipids and/or at least one immobilized hydrophobic agent under conditions enabling binding of the synucleins to the lipids; and in step (c), detecting the lipid-bound proteinase K resistant α-Syn by at least one agent that specifically recognizes and binds the lipid-bound α-Syn, to obtain a PK resistant α-Syn value of the sample. In some specific embodiments, antibodies or aptamers specific for α-Syn are used to determine the amount of the proteinase K resistant α-Syn in the sample. It should be noted that for determining the levels of iron in the sample, any known method or procedure may be used, specifically, the methods described by the examples.

The protein alpha-synuclein or α-synuclein is known to undergo several post-translational modifications (PTM). Several PTMs are enriched within Lewy bodies (LB) and exist at higher levels in α-synucleinopathy brains. Post-translational modifications include but are not limited to phosphorylation, ubiquitination, nitration, sumoylation, acetylation or glycation. Phosphorylation at 5129 (pS129) is one of the main disease-associated α-syn post-translational modifications (PTMs). Still further, α-syn within LBs has been shown to be phosphorylated (at Serine 129, Serine 87, Tyrosine 125, Tyrosine 133 and Tyrosine 136 of said α-Syn), ubiquitinated at lysine residues (K12, K21, or K23), truncated (at its C terminus), and oxidized by tyrosine nitration (Tyrosine 39, Tyrosine 125, Tyrosine 133 and Tyrosine 136 of said α-Syn). It should be understood that in some embodiments, the diagnostic methods of the invention may also use the additional or alternative step of determination of other post translationally modified α-Syn, for the diagnosis of subjects that suffer from at least one synucleopathy and related conditions (e.g., cancer). In some specific embodiments, post translationally modified α-Syn that may be applicable herein may include α-Syn that is modified in at least one amino acid residue, by at least one of phosphorylation, nitration, sumoylation, acetylation and glycation.

In some further embodiments, the methods of the invention may use PSer129 α-Syn and at least one of any of the post translationally modified α-Syn, as combined biomarkers for the diagnosis and detection of at least one synucleopathy.

In some further embodiments, the agent that specifically recognizes and binds the PSer129 α-Syn is at least one of an antibody or any antigen-binding fragment thereof, an aptamer and any combinations thereof. The term “antibody” as used herein, means any antigen-binding molecule or molecular complex that specifically binds to or interacts with a particular antigen. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region (CH). The heavy chain constant region comprises three domains, CH1, CH2 and CH3. Each light chain comprises a light chain variable region (abbreviated herein as LCVR or V_{L}) and a light chain constant region. The light chain constant region comprises one domain (CL1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

Typically, an antibody is composed of two immunoglobulin (Ig) heavy chains and two Ig light chains. In humans, antibodies are encoded by three independent gene loci, namely kappa (κ) chain (Igκ) and lambda (κ) chain (Igλ) genes for the Light chains and IgH genes for the Heavy chains, which are located on chromosome 2, chromosome 22, and chromosome 14, respectively. The antibody used by the method of the invention may be any one of a polyclonal, a monoclonal or humanized antibody or any antigen-binding fragment thereof.

Exemplary categories of antigen-binding domains that can be used in the context of the present invention include antibodies, antigen-binding portions of antibodies, peptides that specifically interact with a particular antigen (e.g., peptibodies), receptor molecules that specifically interact with a particular antigen, proteins comprising a ligand-binding portion of a receptor that specifically binds a particular antigen or antigen-binding scaffolds. The antigen binding domains in accordance with the invention may recognize and bind a specific antigen or epitope, specifically, Ser 129 phosphorylated α-Syn. It should be therefore noted that the term “binding specificity”, “specifically binds to an antigen”, “specifically immuno-reactive with”, “specifically directed against” or “specifically recognizes”, when referring to an antigen or particular epitope, refers to a binding reaction which is determinative of the presence of the epitope in a heterogeneous population of proteins and other biologics.

The term “epitope” is meant to refer to that portion of any molecule capable of being bound by an antibody which can also be recognized by that antibody. Epitopes or “antigenic determinants” usually consist of chemically active surface groupings of molecules such as amino acids or sugar side chains and have specific three dimensional structural characteristics as well as specific charge characteristics. Still further, as indicated above, an “antigen-binding domain” can comprise or consist of an antibody or antigen-binding fragment of an antibody.

Still further, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR)). Other engineered molecules, such as domain-specific antibodies, single domain antibodies, domain-deleted antibodies, chimeric antibodies, CDR-grafted antibodies, bivalent molecules, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}—V_{H}, V_{H}—V_{L} or V_{L}—V_{L} dimers. Alternatively, the antigen-binding fragment of an antibody may contain a monomeric V_{H} or V_{L} domain References to “V_{H}” or a “V_{H}” refer to the variable region of an immunoglobulin heavy chain, including an Fv, scFv, a disulfilde-stabilized Fv (dsFv) or Fab. References to “V_{L}” or a “V_{L}” refer to the variable region of an immunoglobulin light chain, including of an Fv, scFv, dsFv or Fab.

More specifically, the phrase “single chain Fv” or “scFv” refers to an antibody in which the variable domains of the heavy chain and of the light chain of a traditional two chain antibody have been joined to form one chain. Typically, a linker peptide is inserted between the two chains to allow for the stabilization of the variable domains without interfering with the proper folding and creation of an active binding site. A single chain antibody applicable for the invention, e.g., may bind as a monomer. Other exemplary single chain antibodies may form nanobodies, diabodies, triabodies, and tetrabodies.

Other examples of antibody functional fragments include, but are not limited to a single-domain antibody (sdAb) which refers to an antibody fragment consisting of a single monomeric variable antibody domain. The first single-domain antibodies were engineered from heavy-chain antibodies found in camelids; these are called VHH fragments. Cartilaginous fishes also have heavy-chain antibodies (IgNAR, ‘immunoglobulin new antigen receptor’), from which single-domain antibodies called variable new antigen receptor antibody (V-NAR) fragments can be obtained. Thus, the antibody suitable for the invention may also be a variable new antigen receptor antibody (V-NAR). VNARs are a class of small, immunoglobulin-like molecules from the shark immune system. Humanized versions of VNARs could be used to bind protein epitopes that are difficult to access using traditional antibodies.

In some particular embodiments, the antibody suitable for the method and kits of the invention may be any antibody that specifically targets α-synuclein. Non-limiting examples for such antibodies may include MJF-1 (Abcam, Israel), pSyn#64 (WAKO, Japan), Syn303 (Biolegend, ENCO, Israel), MJFR ab (Abcam, Israel), Phospho-alpha Synuclein (Ser129) Polyclonal Antibody (Thermo Fisher Scientific), Phospho-Synuclein-alpha (S129), Antibody PPS091 (R&D SYSTEMS™) or alpha Synuclein (phospho Ser129) antibody (GeneTex), C20 (Santa Cruz); 211 (Santa Cruz); LB509 (Abcam, Israel); Syn-1 (BD Trunsduction); MJF 14-6-4 (Zotal) or any combinations thereof.

In yet some other embodiments the agent that specifically recognizes and binds post translationally modified synuclein, specifically, Ser 129 phosphorylated α-Syn, may be an aptamer. As used herein the term “aptamer” or “specific aptamers” denotes single-stranded nucleic acid (DNA or RNA) molecules which specifically recognizes and binds to a target molecule. The aptamers according to the invention may fold into a defined tertiary structure and can bind a specific target molecule with high specificities and affinities. Aptamers are usually obtained by selection from a large random sequence library, using methods well known in the art, such as SELEX and/or Molinex. In various embodiments, aptamers may include single-stranded, partially single-stranded, partially double-stranded or double-stranded nucleic acid sequences; sequences comprising nucleotides, ribonucleotides, deoxyribonucleotides, nucleotide analogs, modified nucleotides and nucleotides comprising backbone modifications, branch points and non-nucleotide residues, groups or bridges; synthetic RNA, DNA and chimeric nucleotides, hybrids, duplexes, heteroduplexes; and any ribonucleotide, deoxyribonucleotide or chimeric counterpart thereof and/or corresponding complementary sequence. In certain specific embodiments, aptamers used by the invention are composed of deoxyribonucleotides.

Still further, to determine the amount of the PSer129 α-Syn in the sample, a detection step is used to identify the lipid-bound synuclein, using the specific agent that recognizes and bind the bound synuclein. It should be understood that the “detection” step (ii) of the methods of the invention, where PSer129 α-Syn is measured (and step (b) when the total α-Syn is measured or step (c), when the proteinase K resistant α-Syn is measured), involves in some embodiments, the detection of a detectable moiety that is attached or connected either directly or indirectly to the agent (e.g., antibody, aptamer or any combinations thereof), that specifically recognizes and binds translationally modified syn, specifically, PSer129 α-Syn. Still further, as indicated herein, the methods of the invention involve the step of measuring and thereby determining or assessing the signal intensity of the detectable tag or detectable moiety. Reference to “determining”, as used herein, includes estimating, quantifying, calculating or otherwise deriving by measuring an end point indication that may be for example, the appearance of a detectable moiety, any detectable change in the levels or any change in the rate of the appearance or alternatively, the disappearance of the detectable moiety. As used herein, “assessing” refers to quantitative and/or qualitative determination of the detectable moiety attached to the antibodies or aptamers used by the invention, e.g., obtaining an absolute value for the amount or concentration of the antibodies or aptamers, and also of obtaining an index, ratio, percentage, visual or other value indicative of the level of the antibodies or aptamers.

In some further embodiments, the detectable moiety associated directly or indirectly with the antibody or aptamer used by the methods and kits of the invention, may refer to any chemical moiety that can be used to provide a detectable signal, and that can be attached to a nucleic acid or protein via a covalent bond or noncovalent interaction (e.g., through ionic or hydrogen bonding, or via immobilization, adsorption, or the like). Labels generally provide signals detectable by at least one of fluorescence, chemiluminescence, radioactivity, colorimetry, mass spectrometry, X-ray diffraction or absorption, magnetism, enzymatic activity, electrochemical active compounds, or the like. In some specific embodiments, the detectable moiety may be at least one of conductive, electrochemical, fluorescent, chemiluminescent, enzymatic, radioactive, magnetic, metal, and colorimetric label, or any combinations thereof. Examples of labels useful in connection with the invention, include, but are not limited to at least one of haptens, enzymes, enzyme substrates, coenzymes, enzyme inhibitors, fluorophores, quenchers, chromophores, magnetic particles or beads, redox sensitive moieties (e.g., electrochemically active moieties), luminescent markers, radioisotopes (including radionucleotides), conductive materials, or electrochemical materials that in some embodiments may be suitable for electrochemical detection, specifically, nano- and micro-sized materials, such as gold nanoparticles (GNPs), latex, carbon nanotubes (CNTs), graphene (GR), magnetic particles (MBs), quantum dots (QDs) and conductive polymers, biobarcodes and members of binding pairs. More specific examples include at least one of fluorescein, phycobiliprotein, tetraethyl rhodamine, and beta-galactosidase. Binding pairs may include biotin/Strepavidin, biotin/avidin, biotin/neutravidin, biotin/captavidin, GST/glutathione, maltose binding protein/maltose, calmodulin binding protein/calmodulin, enzyme-enzyme substrate, receptor-ligand binding pairs, and analogs and mutants of the binding pairs. It should be appreciated that the use of tags for labeling directly or indirectly the antibody of the invention, is also encompassed by the invention. Non-limiting examples for such tag may include His-tag, Flag, HA, myc and the like. It should be further appreciated that the detectable moieties disclosed herein are applicable for any aspect of the invention. It should be understood that when referring to detectable moiety or tag directly or indirectly attached or connected to the antibody or aptamer used by the invention, the invention relates to antibodies or aptamers that are directly attached to the labeling moiety, or alternatively, indirectly via a linker or via a secondary antibody labelled. In some embodiments, binding of the antibody or aptamer to the lipid-bound PSer129 α-Syn is performed under conditions of physiological pH and physiological concentration of salts.

In yet some further embodiments, the immobilized lipids and/or immobilized hydrophobic agent used by the methods of the invention are synuclein-binding lipids attached or connected directly or indirectly (e.g., via a linker) to a solid support. In more specific embodiments, the lipids comprise at least one of naturally occurring, purified or synthetic phospholipid/s, glycolipid/s, plasmalogen/s, sphingolipid/s, triglycerides, cholesterol, steroids lipoproteins, proteolipids, free fatty acids, eicosanoids and any combinations thereof.

Typically, the immobilized lipids of the invention may be of the kind that present in biological membranes or form membrane structures (including films, micelles, vesicles, liposomes emulsion etc.) in vitro, although when immobilized in the method of the invention they may not present as fully functioning biological membranes. When the lipids are of a natural source, in accordance with a preferred embodiment of the invention, they are of the type that can be found in naturally occurring biological membranes, waxes or oils. Alternatively, synthetic lipids may be used.

In certain embodiments of the invention, the combination of two or more lipids may be used.

In more specific embodiments, the ratio between the different lipids may be respectively from 1:1 to 1:100 specifically 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:25, 1:30, 1:35, 1:40, 1:45, 1:50, 1:55, 1:60, 1:65, 1:70, 1:75, 1:80, 1:85, 1:90, 1:95, or 1:100.

Typically, the membrane forming lipids are naturally occurring, purified or synthetic phospholipids, sphingolipids, plasmalogens, triglycerides, cholesterol, glycolipids or free fatty acids, or eicosanoids a combination of two or more of the above.

Non-limiting examples of naturally occurring or synthetic phospholipids, plasmalogens and ether-phospholipids, sphingolipids, cholesterol and glycolipids may be phosphatidyl inositol, phosphatidyl serine, phosphatidic acid, phosphatdylethanolamine, phosphatidylcholine, phosphatidylserine, phosphatidylglycerol, phosphatidylinositol, phosphoinositides, such as phosphatidylinositol 4, bisphosphate (PI4P) or any PIP of cardiolipin, ceramide, sphingomyelin, glucosylcerebrosidase, galactosylceramide, lactosylceramide, gangliosides, cholesterol, cholesterol-ester, triglycerides, diglycerides and monoglycerides.

The acyl side chains of these lipids may include saturated, unsaturated or poly unsaturated fatty acids. The carbon chain length of the fatty acyl chain may vary between 2-40 carbons for the fatty acid.

In some specific embodiments, the immobilized lipids may comprise a combination of a phosphoinositide (such as phosphatidyl inositol) and phosphatidyl serine. In some embodiments, the immobilized lipids may have acyl side chains such as polyunsaturated fatty acids (PUFAs), or monounsaturated fatty acids (MUFA) or Saturated fatty acid (SFA). In yet some further embodiments, the lipids comprise a combination of two different fatty acids, identical fatty acids or just one fatty acid.

In some specific embodiments, the ratio of the phosphoinositide (such as phosphatidyl inositol) to phosphatidyl serine is from 10:1 to 1:10. In some particular embodiments, the ratio may be 1:2 or 1:1.

In some embodiments, the lipids of the invention may comprise purified myelin and/or cellular membrane fractions and/or purified lipoproteins including apolipoproteins and/or extracts of adipose tissue.

In some embodiments, the lipids may be of an animal, plants, algal, fungal and bacterial sources.

In some specific embodiments, the immobilized lipids of the invention may comprise at least one phosphatidylinositol (PI), phosphatidylserine (PS), phosphatidylethanolamine (PE) and GM-1 ganglioside. In more specific embodiments, the ratio between the above-mentioned lipids may be 1:1:1:1:1.

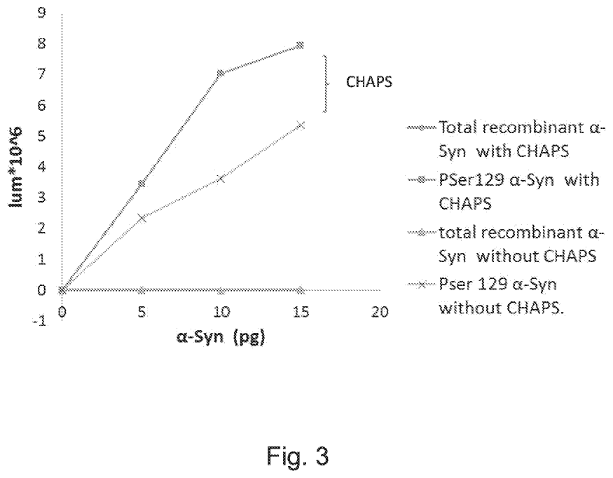

α-Syn binds or interacts with various hydrophobic molecules. In addition, α-Syn binds micelles, a typical form of aggregate in solution, with the hydrophilic “head” regions in contact with surrounding solvent, sequestering the hydrophobic single-tail regions in the micelle centre. Studies have shown a strong association for α-Syn with different hydrophobic molecules that are forming micelles in solution, including micelles formed by detergents. Still further, as shown by FIG. 3 and Example 6, other hydrophobic agents can effectively bind PSer129 α-Syn and may therefore be used, when immobilized to a solid support, to capture PSer129 α-Syn from a sample. As shown by the Examples, PSer129 α-Syn bound to these immobilized hydrophobic agents are recognized by the detecting agent (e.g., an antibody or aptamer). Thus, in some embodiments in addition to or as an alternative to the immobilized lipids used by the methods and kits of the invention, the invention further contemplates the use of any hydrophobic agent. In some embodiments, such hydrophobic agents may be micelle forming detergents, specifically, CHAPS (3-[(3-Cholamidopropyl)dimethylammonio]-1-propane sulfonate hydrate, CHAPS), CHAPSO (3-([3-Cholamidopropyl]dimethylammonio)-2-hydroxy-1-propanesulfonate) and Brij-35 (Brij 35, C₁₂E₂₃, Polyoxyethylene (23) lauryl ether), or any hydrophobic proteins or peptides that are immobilized to a solid support.

As indicated above, the lipids and/or hydrophobic agents used by the methods and kits of the invention are attached or connected directly or indirectly to a solid support. As used herein, “solid support” is defined as any surface to which molecules may be attached through either covalent or non-covalent bonds. Thus, useful solid supports include solid and semi-solid matrixes, such as aero gels and hydro gels, resins, beads, biochips (including thin film coated biochips), micro fluidic chip, a silicon chip, multi-well plates (also referred to as microtiter plates or microplates), membranes, filters, conducting and no conducting metals, glass (including microscope slides) and magnetic supports. More specific examples of useful solid supports include silica gels, polymeric membranes, particles, derivative plastic films, glass beads, cotton, plastic beads, alumina gels, polysaccharides such as Sepharose, nylon, latex bead, magnetic bead, paramagnetic bead, super paramagnetic bead, starch and the like. This also includes, but is not limited to, microsphere particles such as Lumavidin™ or LS-beads, magnetic beads, charged paper, Langmuir-Blodgett films, functionalized glass, germanium, silicon, PTFE, polystyrene, gallium arsenide, gold, and silver. Any other material known in the art that is capable of having functional groups such as amino, carboxyl, thiol or hydroxyl incorporated on its surface, is also contemplated. This includes surfaces with any topology, including, but not limited to, spherical surfaces and grooved surfaces.

It should be further appreciated that any of the lipids and/or hydrophobic agents included in any of the methods and kits of the invention may be provided as lipids embedded, linked, connected, attached, placed or fused to any of the solid support materials described above.

In more specific embodiments, the lipids that may be used by the methods of the invention comprise at least one of phosphatidylinositol (PI), phosphatidylserine (PS), phosphatidylethanolamine (PE), GM-1, GM-2, GM-3, GM-4 gangliosides, phosphatidic acid, phosphatidylcholine, phosphatidylserine, phosphatidylglycerol, phosphatidylinositol, phosphoinositides, cardiolipin, ceramide, sphingomyelin, ether-phospholipids, glucosylcerebrosidase, galactosylceramide lactosylceramide, sphingomyelin, cholesterol, cholesterol-ester, steroids, triglycerides, diglycerides, monoglycerides, fatty acids and eicosanoids, lipoproteins proteolipids and any combinations thereof.

In more specific embodiments, the immobilized lipids applicable in the methods of the invention comprise at least two of naturally occurring, purified or synthetic phosphatidylinositol (PI), phosphatidylserine (PS), phosphatidylethanolamine (PE) and GM-1 ganglioside.

In certain embodiments, the lipids used by the methods and kits of the invention may comprise at least one PI. More specifically, in some embodiments, Phosphatidylinositol (PI) has the following chemical structure, as denoted by Formula I:

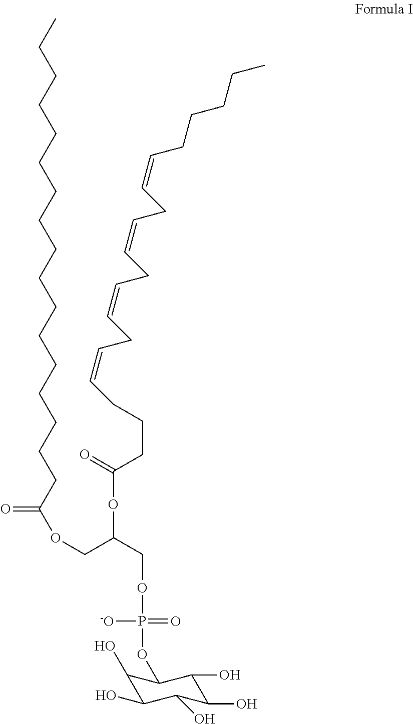

The chemical formula of Phosphatidylinositol (PI) is C₄₇H₈₃O₁₃P and it has a molecular weight of 886.56 g/mol (neutral with fatty acid composition—18:0, 20:4).

In yet some further embodiments, the lipids used by the methods and kits of the invention may comprise at least one PS. In more specific embodiments, Phosphatidylserine (PS) has the following chemical structure, as denoted by Formula II:

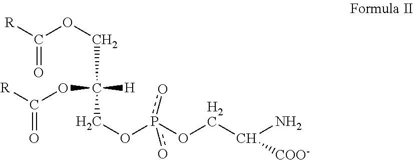

The chemical formula of Phosphatidylserine (PS) is C₁₃H₂₄NO₁₀P and it has a molecular weight of 385,304 g/mol. Still further, in certain embodiments, the lipids used by the methods and kits of the invention may comprise at least one PE. More specifically, Phosphatidylethanolamine (PE) has the following chemical structure, as denoted by Formula III:

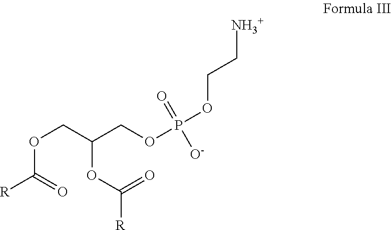

The chemical formula of Phosphatidylethanolamine (PE) is C₄₀H₈₀NO₈P and it has a molecular weight of 734,0389±0,0402 g/mol.

In yet some further embodiments, the lipids used by the methods and kits of the invention may comprise GM-1 ganglioside. In certain embodiments, GM-1 ganglioside has the following chemical structure, as denoted by Formula IV:

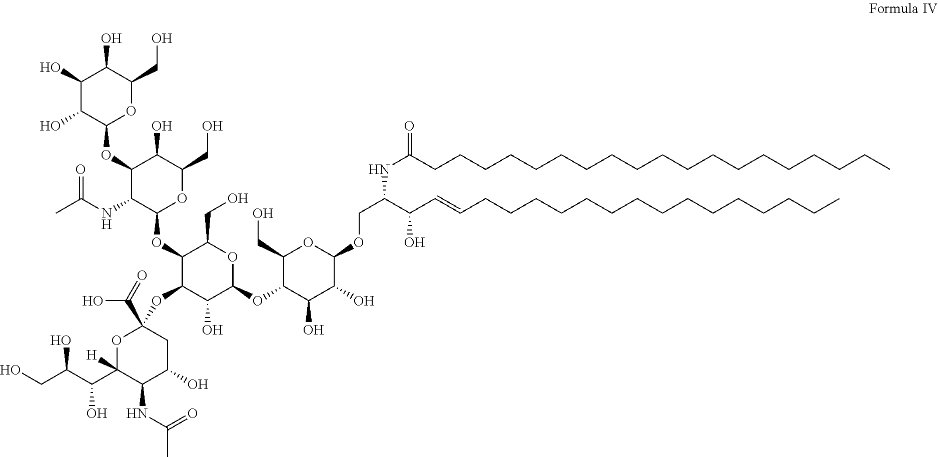 [SMILES: CCCCCCCCCCCCCCC/C=C/[C@H](O)[C@H](CO[C@@H]1O[C@H](CO)[C@@H](O[C@@H]2O[C@H](CO)[C@H](O[C@@H]3O[C@H](CO)[C@H](O)[C@H](O[C@@H]4O[C@H](CO)[C@H](O)[C@H](O)[C@H]4O)[C@H]3NC(C)=O)[C@H](O[C@]3(C(=O)O)C[C@H](O)[C@@H](NC(C)=O)[C@H]([C@H](O)[C@H](O)CO)O3)[C@H]2O)[C@H](O)[C@H]1O)NC(=O)CCCCCCCCCCCCCCCCCCC]

The chemical formula of GM-1 ganglioside is C₇₇H₁₃₉N₃O₃₁ and it has a molecular weight of 1602.949 g/mol.

In some further embodiments, the lipids suitable for the methods of the invention may comprise at least one phospholipids. Phospholipids are a class of lipids that are a major component of all cell membranes. They can form lipid bilayers because of their amphiphilic characteristic. The structure of the phospholipid molecule generally consists of two hydrophobic fatty acid “tails” and a hydrophilic “head” consisting of a phosphate group. The two components are joined together by a glycerol molecule. The phosphate groups can be modified with simple organic molecules such as choline, ethanolamine or serine.

Phosphololipids may have diacylglyceride structures (glycerophospholipid) and may comprise Phosphatidic acid (phosphatidate) (PA), Phosphatidylethanolamine (PE), Phosphatidylcholine (lecithin) (PC), Phosphatidylserine (PS), or Phosphoinositides.

Examples of Phosphoinositides include but are not limited to Phosphatidylinositol (PI), Phosphatidylinositol phosphate (PIP), Phosphatidylinositol bisphosphate (PIP2) and Phosphatidylinositol trisphosphate (PIP3).

Phospholipids may also refer to Phosphosphingolipids (Sphingolipid) such as Ceramide phosphorylcholine (Sphingomyelin) (SPH), Ceramide phosphorylethanolamine (Sphingomyelin) (Cer-PE) or Ceramide phosphoryllipid.

“Phospholipids” comprises phosphatidic acids, phosphoglycerides, and phosphosphingolipids. Phosphatidic acids comprise a phosphate group coupled to a glycerol group, which may be monoacylated or diacylated. Phosphoglycerides (or glycerophospholipids) include a phosphate group intermediate an organic group (e.g., choline, ethanolamine, serine, inositol) and a glycerol group, which may be monoacylated or diacylated. Phosphosphingolipids (or sphingomyelins) include a phosphate group intermediate an organic group (e.g., choline, ethanolamine) and a sphingosine (non-acylated) or ceramide (acylated) group. The term “phospholipid” also refers to salts (e.g., sodium, ammonium) of phospholipids. For phospholipids that include carbon-carbon double bonds, individual geometrical isomers (cis, trans) and mixtures of isomers are included. Non-limiting examples of phospholipids include phosphatidylcholines, phosphatidylethanolamines (PE), phosphatidylglycerols, phosphatidylserines, phosphatidylinositols, and phosphatidic acids, and their lysophosphatidyl (e.g., lysophosphatidylcholines and lysophosphatidylethanolamine) and diacyl phospholipid (e.g., diacylphosphatidylcholines, diacylphosphatidylethanolamines, diacylphosphatidylglycerols, diacylphosphatidylserines, diacylphosphatidylinositols, and diacylphosphatidic acids) counterparts. In some embodiments, the acyl groups of the phospholipids are the same. In other embodiments, the acyl groups of the phospholipids are different. In some embodiments, the acyl groups are derived from fatty acids having C10-C24 carbon chains (e.g., acyl groups such as lauroyl, myristoyl, palmitoyl, stearoyl or oleoyl groups). Representative diacylphosphatidylcholines (i.e., 1,2-diacyl-sn-glycero-3-phosphocholines) include distearoylphosphatidylcholine (DSPC), dioleoylphosphatidylcholine (DOPC), dipalmitoylphosphatidylcholine (DPPC), dilinoleoylphosphatidylcholine DLPC), palmitoyloleoylphosphatidylcholine (POPC), palmitoyllinoleoylphosphatidylcholine, stearoyllinoleoylphosphatidylcholine stearoyloleoylphosphatidylcholine, stearoylarachidoylphosphatidylcholine, didecanoylphosphatidylcholine (DDPC), dierucoylphosphatidylcholine (DEPC), dilinoleoylphosphatidylcholine (DLOPC), dimyristoylphosphatidylcholine (DMPC), myristoylpalmitoylphosphatidylcholine (MPPC), myristoylstearoylphosphatidylcholine (MSPC), stearoylmyristoyl-phosphatidylcholine (SMPC), palmitoylmyristoylphosphatidylcholine (PMPC), palmitoylstearoylphosphatidylcholine (PSPC), stearoylpalmitoylphosphatidylcholine (SPPC), and stearoyloleoylphosphatidylcholine (SOPC). Examples of diacylphosphatidylethanolamines (i.e., 1,2-diacyl-sn-glycero-3-phosphoethanolamines) include, but are not limited to, dioleoylphosphatidylethanolamine (DOPE), dipalmitoylphosphatidylethanolamine (DPPE), distearoylphosphatidylethanolamine (DSPE), dilauroylphospha-tidylethanolamine (DLPE), dimyristoylphosphatidylethanolamine (DMPE), dierucoylphosphatidylethanolamine (DEPE), and palmitoyloleoylphospha-tidylethanolamine (POPE). Examples of diacylphosphatidylglycerols (i.e., 1,2-diacyl-sn-glycero-3-phosphoglycerols) include, but are not limited to, dioleoylphosphatidylglycerol (DOPG), dipalmitoylphosphatidylglycerol (DPPG), dierucoylphosphatidylglycerol (DEPG), dilauroylphosphatidylglycerol (DLPG), dimyristoylphosphatidylglycerol (DMPG), distearoylphosphatidylglycerol (DSPG), and palmitoyloleoylphospha-tidylglycerol (POPG).

Examples of diacylphosphatidylserines (i.e., 1,2-diacyl-sn-glycero-3-phosphoserines) include, but are not limited to, dilauroylphosphatidylserine (DLPS), dioleoylphosphatidylserine (DOPS), dipalmitoylphosphatidylserine (DPPS), and distearoylphosphatidylserine (DSPS).

Examples of diacylphosphatidic acids (i.e., 1,2-diacyl-sn-glycero-3-phosphates) include, but are not limited to, dierucoylphosphatidic acid (DEPA), dilauroylphosphatidic acid (DLPA), dimyristoyiphosphatidic acid (DMPA), dioleoylphosphatidic acid (DOPA), dipalmitoylphosphatidic acid (DPPA), and distearoylphosphatidic acid (DSPA).

Examples of phospholipids include, but are not limited to, phosphosphingolipids such as ceramide phosphoryllipid, ceramide phosphorylcholine, and ceramide phosphorylethanolamine. In another embodiments, the lipids of the invention may comprise β-glycolipids.

Types of β-glycolipid include and are not limited to a glucosylceramide, a monosaccharide ceramide, a galatosylceremide, a lactosyl-ceramide, a gal-gal-glucosyl-ceramide, GM-1 ganglioside, GM2 ganglioside, GM3 ganglioside, or globoside. It should be appreciated that any of the lipids and/or hydrophobic agents disclosed herein, may be used and comprised by any of the methods and kits of the invention.

In yet some further embodiments, prior to attachment to a solid support, the lipids used in the methods and kits of the invention are dissolved in at least one organic solvent. The term “organic solvent” as used herein refers to a large group of chemical compounds capable of dissolving nonwater-soluble materials such as fats, oils, waxes, resins, rubber, asphalt, cellulose filaments, and plastic materials. Non-limiting examples of organic solvents include ethanol, acetic acid, acetone, acetonitrile, benzene, 1-butanol, 2-butanol, 2-butanone, t-butyl alcohol, carbon tetrachloride, chlorobenzene, chloroform, cyclohexene, cyclohexane, 1,2-dichloroethane, diethylene glycol, ether, diethyl ether, diglyme (diethylene glycol, dimethyl ether), 1,2-dimethoxyethane (glyme, DME), dimethylformamide (DMF), dimethyl sulfoxide (DMSO), 1,4-dioxane, ethanol, ethyl acetate, ethylene glycol, glycerin, heptane, hexamethylphosphoramide (HMPA), hexamethylphosphorous, triamide (HMPT), hexane, methanol, methyl t-butyl ether (MTBE), methylene chloride, N-methyl-2-pyrrolidinone (NMP), nitromethane, pentane, petroleum ether (ligroine), isopropanol, 1-propanol, 2-propanol, pyridine, tetrahydrofuran (THF), toluene, triethyl amine, o-xylene, m-xylene, or p-xylene. In more specific embodiments, the organic solvent is at least one of methanol, cyclohexene, ethanol, chloroform, ether, isopropanol, and any combinations thereof. In some particular embodiments, the organic solvent may be methanol or cyclohexene. In more specific embodiments, the organic solvent may be cyclohexene.

As shown by Example 2, the use of cyclohexene to dissolve the lipids before attachment to a solid support increase the sensitivity of the assay. Therefore, in some embodiments, for detection and determining the amount of PSer129 α-Syn, the immobilized lipids are dissolved in cyclohexene prior to their attachment to a solid support. In yet some further embodiments, to increase specificity, for determining the total amount of α-Syn in a sample, the immobilized lipids are dissolved in methanol prior to their attachment to a solid support.

The invention provides powerful methods for diagnosis and/or detection of synucleinopathies. “Alpha-synuclein pathology disorders” or “Synucleinopathies” is used to name a group of neurodegenerative disorders characterized by fibrillary aggregates of alpha-synuclein protein in the cytoplasm of selective populations of neurons and glia. More specifically, as used herein are disorders characterized by the presence of a specific intracellular protein aggregates (inclusion bodies) known as Lewy bodies that contain mainly alpha-synuclein protein. Alpha-synuclein protein is found naturally as an unfolded cytoplasmic protein in neuronal synaptic areas.

Overexpression of alpha-synuclein interrupts normal cell functions and leads to decreases in neurite outgrowth and cell adhesion. Alpha-synuclein aggregates comprising monomeric, oligomeric intermediate, or fibrillar forms are thought to be involved in a critical step in the pathogenesis of Parkinson's disease (PD) and in other alpha-synucleinopathies, such as multiple system atrophy (MSA) and dementia with Lewy bodies (DLB). These chronic neurodegenerative diseases of the CNS are characterized by the development of Lewy bodies containing alpha-synuclein protein. Oligomeric and monomeric alpha-synuclein have both been detected in cerebrospinal fluid and plasma samples from PD patients, suggesting that small aggregates of alpha-synuclein access the extracellular space. Previous animal and clinical data suggest that misfolded alpha-synuclein can be released from cells by exocytosis and transmitted from one brain area to another via cell-to cell propagation. Although the exact mechanism of alpha-synuclein transmission remains unknown, evidence suggests that clathrin-mediated endocytosis (CME) may have an important role in internalization of extracellular alpha-synuclein. As the cargo protein for endocytosis is usually recognized by a specific receptor on the cell surface, it is possible that alpha-synuclein may interact with cell-surface receptors that have not been well specified until now. N-methyl-D-aspartate (NMDA) receptor subunits contain motifs that bind the endocytic adaptor protein involved in CME. Additionally, a recent study provided the evidence that alpha-synuclein could promote endocytic internalization of surface NMDA receptors through a mechanism requiring clathrin, suggesting an interaction between alpha-synuclein and NMDA receptors. Accordingly, alpha-synuclein propagation from one area of the brain to others via cell-to-cell transmission is closely related with disease progression or clinical severity. Still further, Lewy body pathology in Parkinson's disease also found in peripheral nervous system. In neurons innervating the gastrointestinal tract and appendix. Peripheral Lewy pathology is suggested to precede the CNS Lewy pathology and according to Braak hypothesis, precede disease onset. Therefore, peripheral Lewy pathology may represent an early stage of the disease, or prodromal stage. It should be understood that this may be relevant for early diagnosis of the disease, before pathology occurs in the brain. More specifically, as the assay of the invention is a blood test and synucleopathic disease begins at the periphery, detection of blood cell PSer129 α-Syn, enables in some embodiments of the invention, early, pre-symptomatic diagnosis of PD patients. In some specific embodiments, such synucleinopathy is at least one of Parkinson's disease (PD), Lewy body dementia (LBD) and multiple system atrophy (MSA). It should be noted that in some embodiments, the invention may be further applicable for any synucleinopathies, any stage thereof (either early or advanced), and any symptoms, disorders and conditions associated therewith.

In some embodiments, the methods and kits of the invention may be applicable for diagnosing and/or detecting PD. “Parkinson's disease (PD)” as used herein, is a neurodegenerative disease resulting from degeneration of midbrain dopamine neurons and accumulation of alpha-synuclein containing Lewy bodies in surviving neurons. The diagnosis of PD is based on the presence of cardinal motor features in the absence of other aetiological conditions. These motor features include the classical triad of bradykinesia, a resting pill-rolling tremor, and rigidity typically in association with hypomimia, hypophonia, micrographia and postural instability. Non-motor features of PD may even precede its diagnosis, constituting prodromal or premotor PD. These premotor features include problems with olfaction, constipation, mood and sleep, and following the clinical diagnosis of PD, they can become more prominent. Cognitive problems and dementia also commonly develop in PD, affecting almost 50% by 10 years from diagnosis. However, in some individuals with an alpha-synucleinopathy, significant cognitive problems precede the onset of parkinsonian motor symptoms, and these cases are clinically classified with a diagnosis of Dementia with Lewy Bodies. There is clearly a major degree of overlap between these two conditions both clinically and pathologically, but at present, the clinical distinction rests on the time interval between the onset of motor symptoms and dementia, with a minimum one year interval being required for a diagnosis of PD as opposed to Lewy body dementia (DLB).

Multiple system atrophy (MSA) is the rarest of the three major alpha synucleinopathies and differs significantly from PD and DLB in terms of its clinical presentation and its more aggressive course, reflecting differences in the underlying neuroanatomical pathways involved. In some embodiments, the methods of the invention are particularly useful for diagnosis and detection of PD. As detecting a peripheral disease as indicated above, the methods and kits of the invention may be applicable for early detection of PD (detection at early stages of the disease). It is important to note that the interest in PSer129 α-Syn as a pathogenic form of α-Syn is based on its association with α-Syn toxicity in the nervous system. However, the biological significance of phosphorylation at Serine 129 on blood cells-expressed α-Syn was not clear, since no PSer129 α-Syn was reported in blood samples. The present invention now shows that PD sub groups that differ in presentation of cognitive symptoms could be differentiated based on their levels of blood cells-expressed α-Syn. The levels determined for total and PK^{res} α-Syn significantly differ between a PD group presenting cognitive impairment (PD-D) and a PD group with motor symptoms only. In addition, PSer129 α-Syn levels show a tendency for a significant difference between these sub groups.

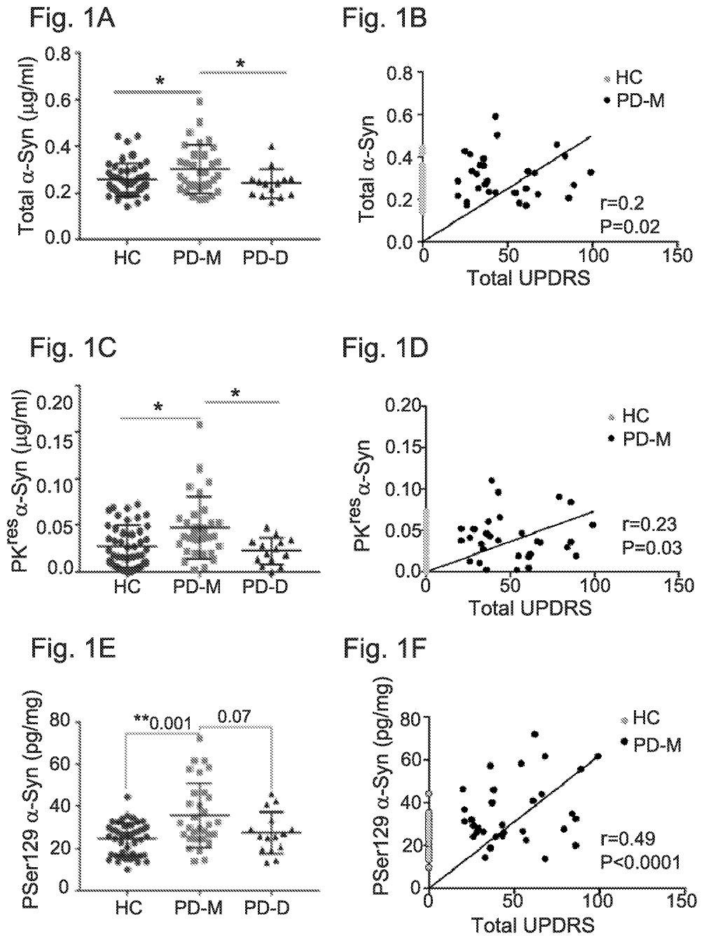

More specifically, as shown by FIGS. 1 and 2, the methods and kits of the invention may be specifically applicable or detecting PD patients with motor progression (PD-M). Thus, in some embodiments, a positive Sum value calculated in the sample, as defined in step (d) where all four parameters are determined, or by step (b), when only PSer129 α-Syn value is determined, indicates that the tested subject suffers from PD-M. More specifically, the methods of the invention specifically distinguish PD-M patients from healthy subjects and more importantly, from PD patients with dementia, PD-D. Thus, in some embodiments, the invention provided specific diagnostic and prognostic methods and kits applicable for PD-M patients. More specifically, the motor course of PD, also termed as PD-motor (PD-M) or Motor progression, often follows a predictable course with patients initially responding well to dopaminergic medication for a number of years.

Still further, it should be understood that in some embodiments, the diagnostic and prognostic methods and kits of the invention may be applicable also for diagnosis, and optionally, early diagnosis of subjects in risk for developing PD. In more specific embodiments, a subject at risk for developing PD may be for example a subject that carry at least one mutation in at least one gene encoding a synuclein protein, specifically, at least one of alpha-, beta- and gamma- syn, or in a gene encoding any protein associated therewith, specifically any protein that is associated or connected with syn stability, expression or activity. In yet some further embodiments, a subject that overexpresses the synuclein genes due to biochemical or genetic reasons.

In yet some further embodiments, a subject at risk for developing PD may be subjects that carry at least one mutation in the gene (PARK8 gene) encoding the Leucine-rich repeat kinase 2 (LRRK2) protein, also known as dardarin, or in a gene encoding any protein associated therewith, specifically any protein that is associated or connected with LRRK2 stability, expression or activity. In yet some further embodiments, a subject at risk for developing PD may be a subject that carry at least one mutation in at least one gene encoding glucocerebrosidase (GBA) or in a gene encoding any protein associated therewith, specifically any protein that is associated or connected with GBA stability, expression or activity, may be also considered as at risk for developing PD in accordance with the invention.

In yet some further embodiments, the diagnostic and prognostic methods and kits of the invention may be applicable for DLB. More specifically, “Dementia with Lewy Bodies (DLB)”, as used herein, is a relatively common cause of dementia, estimated to account for up to 30% of dementia cases, and affecting up to 5% of those over the age of 75. Pathologically, it is defined by the presence of alpha synuclein containing Lewy bodies in the brain, but their distribution differs from that in PD, affecting the neocortex, limbic system and brainstem, in contrast to the nigrostriatal and brainstem-predominant pattern seen in early PD.

In yet some further embodiments, the diagnostic and prognostic methods and kits of the invention may be applicable for MSA. “Multiple system atrophy (MSA)”, as used herein, is much rarer than PD with an estimated prevalence of 4.4 per 100 000 (PD is around 45 times more common).

Still further, in some embodiments, the diagnostic and/or prognostic methods and kits of the invention may be applicable for “Alzheimer's disease (AD)”. In yet some further embodiments, the diagnostic and/or prognostic methods and kits of the invention may be applicable for Niemann-pick-type 1, and neuro-degeneration with brain iron accumulation-1.

The α-synuclein protein is linked to various cancers although the biological consequences are relatively unknown Immuno-histological studies revealed its expression in ependymoma, astrocytoma, breast and ovarian cancerous tissues, and these cancers co-occur with PD in epidemiological studies discussed previously (Fung K M, et al. (2003) Acta Neuropathol; 106(2):167-75). The methylated state of SNCA and the presence of α-synuclein in melanocytic lesions may be used as biomarkers for some lymphomas and melanoma (Welinder C, et al. (2014) PLoS One; 9(10):e110804). Although α-synuclein is a hallmark of PD, its propagation mechanism may help explain its appearance in cancers outside the brain and in human plasma and cerebrospinal fluid (Allsop D et al. (2006) FASEB J; 20(3):419-25).

In melanoma, S129-phosphorylated form of α-synuclein, the pathological form in Lewy bodies of PD, but not the unphosphorylated form, is localized to the surface of melanoma cells and their released microvesicles' membranes (Kamitani T, et al. (2013) J Cell Sci; 126(Pt 2):696-704).

Thus, in some embodiments thereof, the diagnostic and prognostic methods and kits of the invention may be applicable for the diagnosis of any cancer related to synucleinopathy. The term “cancer” is used herein interchangeably with the term “tumor” and denotes a mass of tissue found in or on the body that is made up of abnormal cells. In the context of cancer, methods of the present invention are particularly applicable to various types of melanoma or any other type of skin cancer [e.g., Basal cell carcinoma. Squamous cell carcinoma, Actinic keratosis (solar keratosis)], breast, prostate, colon, brain, kidney and lung cancers, and any further carcinoma, lymphoma, sarcoma, leukemia of various stages, grades and primary origins.

Still further, in some embodiments, the diagnostic methods of the invention may further comprise a therapeutic step for the diagnosed patients. Thus, in certain embodiments, the method of the invention further comprises the step of administering to a subject diagnosed with the at least one synucleinopathy, for example, PD, a therapeutically effective amount of a therapeutic agent for the diagnosed synucleinopathy. It should be appreciated that in some embodiments of the invention, such therapeutic agents may be also included in the kits of the invention.

Thus, in some embodiments, any of the diagnostic and prognostic methods disclosed by the invention may further comprise an additional therapeutic step that involves the administration of a suitable therapeutic agent to a subject diagnosed or classified by the method of the invention as a subject suffering from the particular synucleinopathy or cancer. In some embodiments, where the diagnosed subject is classified or identified by the methods of the invention as a subject suffering from PD, the diagnostic method of the invention further comprises the step of administering at least one PD therapeutic agent to a subject diagnosed with PD. To date, there is no cure for the Parkinson's disease condition, but there are medications and therapies available to address some of the symptoms and improve quality of life for patients. Non limiting examples of available treatments of Parkinson's disease are Carbidopa-levodopa, Carbidopa-levodopa infusion (Duopa), Dopamine agonists, monoamine oxidase B (MAO-B) inhibitors, Catechol-O-methyltransferase (COMT) inhibitors, Anticholinergics, Amantadine or deep brain stimulations. It should be understood that the assay of the invention is particularly applicable for the detection or diagnosis of a disease, for determining the severity of diseases, for drug development or for monitoring a therapeutic regime. The severity of the disease being diagnosed and/or the progress of a therapeutic regime being monitored are determined by the use of calibration curves generated with known amounts of purified, recombinant PSer129 α-Synclein, or by the use of calibration curves obtained with data obtained from healthy control subjects and/or patients having the relevant disease. Or is addition to clinical data or clinical diagnosis.

In yet some further embodiments, the methods of the invention as described herein are useful for determining the severity and progression of the at least one synucleinopathy in a diagnosed subject. Thus, the calculated sum of the diagnosed subject in samples obtained from patients with known severity of the specific synucleinopathy or specific cancer, are used as standard values that are compared with the calculated values of the diagnosed subject. It should be further noted that the methods of the invention may be particularly useful for determining the severity and progression of patients diagnosed with PD-M. In accordance with a further embodiment of the invention, the method is used for monitoring the occurrence and progression of the disease in peripheral tissues, such as the gastro-intestinal system or heart.

As shown by FIGS. 1E and 2A, the methods of the invention distinguish between PD patients that develop motor symptoms, and PD patients that develop dementia. Thus, in some embodiments, the invention further provides a powerful tool for assessing and/or predicting if a subject diagnosed with PD, will develop dementia. This assessment is achieved by any of the methods and kits of the invention. In such case, a positive Sum value calculated in the sample, as defined in step (d) where all four parameters are determined, or by step (b), when only PSer129 α-Syn value is determined, indicates that the tested subject suffers from PD-M with no dementia. In other words, a negative Sum value calculated in the sample, indicates that the tested subject is a PD subject that is likely to develop dementia (PD-D).

In yet some further embodiments, the method of the invention may be useful for monitoring and assessing responsiveness of a mammalian subject suffering from at least one synucleinopathy to a treatment regimen. In more specific embodiments, the method comprises the steps of:

In a first step (a), determining the amount of PSer129 α-Syn in at least one whole blood sample of the diagnosed subject, or at least one aliquot thereof; to obtain a PSer129 α-Syn value of the sample. In yet some optional step (a), this step further involves determining at least one of: (i) the total α-Syn amount, to obtain a total α-Syn value of the sample; (ii) the amount of proteinase K-resistant α-Syn, to obtain a PK resistant α-Syn value of the sample; and (iii) the iron level, to obtain an iron value of the sample.

The next step (b), is particularly relevant if the values of the additional parameters are determined, specifically, total α-Syn value, PK resistant α-Syn value and the iron value of the sample are determined. In that case, step (b) involves calculating the weighed sum of said PSer129 α-Syn value as determined in step (a), and of at least one of the α-Syn value as determined in step (a i), the PK resistant α-Syn value as determined in step (a ii), and the iron value as determined in step (a iii), to obtain a Sum value of the sample;

The next step (c), involves repeating step (a) and optionally step (b), to obtain a Sum value for at least one more temporally-separated sample.

In the next step (d), calculating the rate of change of said Sum values between the temporally-separated samples to obtain a rate of change Sum value; and

In the next step (e), determining if the rate of change Sum value obtained in step (d) is positive or negative with respect to a predetermined standard rate of change Sum value or to the rate of change Sum value calculated in at least one control whole blood sample.

It should be noted that in some embodiments, a positive rate of change Sum value indicates that the subject responds to the therapeutic regimen.

In yet some other embodiments, the invention further provides a method for assessing responsiveness of a mammalian subject to treatment with a specific therapeutic agent or evaluating and/or monitoring the efficacy of treatment on a subject. This method is based on determining the post translationally modified α-Syn values of the invention before and any time after initiation of treatment, and calculating the rate of the change in said values as a result of the treatment. In some specific embodiments, at least two samples are obtained from a subject diagnosed with the specific synucleopathy, specifically, PD. In yet some further embodiments at least one of the temporally separated samples is obtained prior to treatment. In yet some further embodiments, at least one of the temporally separated samples is obtained after the initiation of the treatment. Still further, the Sum value as discussed above, or alternatively, the PSer129 α-Syn value is calculated or determined, respectively, for each sample. In some specific embodiments, to evaluate if a patient is responding to a specific treatment regimen, for example, PD-M patients, the Sum values calculated for at least one sample obtained after the initiation of the treatment is subtracted from the Sum value obtained before the initiation of the treatment, or from a sample obtained from an earlier stage of the treatment. In some embodiments, where the difference in the Sum values of the samples is positive, specifically, where the difference, or the “rate of change” as used herein, is larger than—or equal to—0, the subject is classified as a responder. In yet some further embodiments, where the difference is lower than 0, the subject is determined as having a “negative” rate of change and is therefore classified as a non-responder.

In yet some further alternative embodiments, the rate of change may be calculated by any alternative way or by using any tool, e.g., by calculating the ratio between the samples (e.g., dividing the Sum values of a first sample with the other sample).

As indicated above, in accordance with some embodiments of the invention, in order to assess the patient condition, or monitor the disease progression, as well as responsiveness to a certain treatment, at least two “temporally-separated” test samples must be collected from the examined patient and compared thereafter in order to obtain the rate of change in the value of at least one of the post translationally modified α-Syn proteins between said samples. In practice, to detect a change in at least one of these parameters between said samples, at least two “temporally-separated” test samples and preferably more must be collected from the patient. The value is then determined using the method of the invention, applied for each sample. As detailed above, the rate of change in parameters is calculated by determining the ratio between at least two values obtained from the same patient in different time-points or time intervals.

This period of time, also referred to as “time interval”, or the difference between time points (wherein each time point is the time when a specific sample was collected) may be any period deemed appropriate by medical staff and modified as needed according to the specific requirements of the patient and the clinical state he or she may be in. For example, this interval may be at least one day, at least three days, at least three days, at least one week, at least two weeks, at least three weeks, at least one month, at least two months, at least three months, at least four months, at least five months, at least one year, or even more.

In some embodiments, one of the time points may correspond to a period in which a patient is experiencing a remission of the disease.

When calculating the rate of change, one may use any two samples collected at different time points from the patient. To ensure more reliable results and reduce statistical deviations to a minimum, averaging the calculated rates of several sample pairs is preferable. A calculated or average value of a positive rate of change of the calculated Sum value indicates that said subject exhibits a beneficial response to said treatment; thereby monitoring the efficacy of a treatment with a therapeutic agent and the disease progression. It should be noted that in certain embodiments, where normalization step is being performed, the values referred to above, are normalized values.

The number of samples collected and used for evaluation of the subject may change according to the frequency with which they are collected. For example, the samples may be collected at least every day, every two days, every four days, every week, every two weeks, every three weeks, every month, every two months, every three months every four months, every 5 months, every 6 months, every 7 months, every 8 months, every 9 months, every 10 months, every 11 months, every year or even more. Furthermore, to assess the trend in level rates according to the invention, it is understood that the rate of change may be calculated as an average rate of change over at least three samples taken in different time points, or the rate may be calculated for every two samples collected at adjacent time points. It should be appreciated that the sample may be obtained from the monitored patient in the indicated time intervals for a period of several months or several years. More specifically, for a period of 1 year, for a period of 2 years, for a period of 3 years, for a period of 4 years, for a period of 5 years, for a period of 6 years, for a period of 7 years, for a period of 8 years, for a period of 9 years, for a period of 10 years, for a period of 11 years, for a period of 12 years, for a period of 13 years, for a period of 14 years, for a period of 15 years or more. In one particular example, the samples are taken from the monitored subject every two months for a period of 5 years.

As indicated above, the invention provides diagnostic and prognostic methods. “Prognosis” is defined as a forecast of the future course of a disease or disorder, based on medical knowledge. This highlights the major advantage of the invention, namely, the ability to predict progression of the disease, based on the expression value of the post translationally modified α-Syn proteins, specifically, PSer129 α-Syn.

The method for monitoring disease progression or early prognosis for disease relapse as detailed herein may be used for personalized medicine, by collecting at least two samples from the same patient at different stages of the disease.

A useful biomarker assists diagnosis of PD. Moreover, the development of a useful biomarker will benefit better classification of the disease. PD may potentially consist of subtypes, including variable symptoms, response to therapy, rate of disease progression and genetics. A specific biomarker as shown by the invention, can present compatibility with a specific disease subtype. It is therefore plausible that testing the diagnostic efficacy of total α-Syn and its post translational modified forms, in blood samples of patients with respect to the synuclein burden in the brain can further improve the outcome of the biomarker of the invention. It should be therefore appreciated that the invention further encompasses the combination of any of the methods discussed herein that are performed in blood samples, with any other diagnostic methods for detection of synucleinopathies, specifically, PD, more specifically, PD-M.

In another aspect, the present invention is directed to a kit containing some or all of the substances, components and agents required to perform the methods of the invention and any technique disclosed hereinabove. Such kits may be used to measure the concentration of PSer129 synucleins in a sample. The kits of the present invention further comprise additional means, reagents and buffers that are needed to perform any of the assays disclosed by the invention, and may optionally also comprise a set of instructions for using said kit. More specifically, a further aspect of the invention relates to a kit comprising:

(a) immobilized lipids and/or at least one immobilized hydrophobic agent; and (b) at least one agent that specifically recognizes and binds PSer129 α-Syn. In some embodiments, the kit of the invention may optionally further comprise at least one of:
(c) at least one agent that specifically recognizes and binds α-Syn; (d) Proteinase K;
(e) means for determining iron levels in a sample; (f) pre-determined calibration curve providing standard; (g) at least one control sample; (h) at least one means for depleting hemoglobin from a whole blood sample; and (i) at least one organic solvent.

In some embodiments, the kit of the invention may be particularly applicable for whole blood samples that comprise blood cells.

In more specific embodiments, the whole blood sample is a hemoglobin depleted sample.

In yet some further alternative embodiments, the kits of the invention may further comprise reagents for determining other post translationally modified α-Syn forms in a blood sample, for example, α-Syn modified in at least one amino acid residue, by at least one of phosphorylation, nitration, sumoylation, acetylation and glycation. In more specific embodiments of the kit of the invention such α-Syn phosphorylation may be in at least one of Serine 129, Serine 87, Tyrosine 125, Tyrosine 133 and Tyrosine 136 of said α-Syn, or any combinations thereof. In yet some further embodiments, such α-Syn may be modified by nitration in at least one of Tyrosine 39, Tyrosine 125, Tyrosine 133 and Tyrosine 136 of said α-Syn.

In yet some further specific embodiments, the kits of the invention comprises at least one agent that specifically recognizes and binds the PSer129 α-Syn. In more specific embodiments, such agent is at least one of an antibody or any antigen-binding fragment thereof, an aptamer and any combinations thereof. Still further, in some embodiments, the immobilized lipids comprised within the kits of the invention are synuclein-binding lipids attached or connected directly or indirectly to a solid support. In more specific embodiments, such lipids comprise at least one of naturally occurring, purified or synthetic phospholipid/s, glycolipid/s, plasmalogen/s, sphingolipid/s, triglycerides, cholesterol, steroids, glycolipid/s, lipoproteins, proteolipids, free fatty acids, eicosanoids and any combinations thereof.

Still further, in some embodiments, the lipids of the kits of the invention comprise at least one of phosphatidylinositol (PI), phosphatidylserine (PS), phosphatidylethanolamine (PE), GM-1, GM-2, GM-3, GM-4 gangliosides, phosphatidic acid, phosphatidylcholine, phosphatidylserine, phosphatidylglycerol, phosphatidylinositol, phosphoinositides, cardiolipin, ceramide, sphingomyelin, ether-phospholipids, glucosylcerebrosidase, galactosylceramide lactosylceramide, sphingomyelin, cholesterol, cholesterol-ester, steroids, lipoproteins, proteolipid, triglycerides, diglycerides, monoglycerides, and any combinations thereof. In certain embodiments, the immobilized lipids of the kits of the invention comprise at least two of naturally occurring, purified or synthetic phosphatidylinositol (PI), phosphatidylserine (PS), phosphatidylethanolamine (PE) and GM-1 ganglioside. Still further, in some embodiments, the lipids of the kits of the invention are dissolved in at least one organic solvent prior to attachment to the solid support. In more specific embodiments, the organic solvent used to dissolve the lipids of the kits of the invention may be at least one of ethanol, cyclohexene, chloroform, methanol, ether, isopropanol, and any combinations thereof. In some embodiments, the kits of the invention are adapted to perform any of the methods for assaying post translationally modified α-Syn, specifically, PSer129 α-Syn in a sample as defined by the invention. In yet some further specific embodiments, the kits of the invention may be particularly useful for the detection or diagnosis of at least one synucleinopathy in a subject, or any related conditions, disorders or symptoms. In more specific embodiments, the kits of the invention may be applicable for performing any of the methods of the invention as described herein above. In more specific embodiments, the kits of the invention may be applicable for the early detection or diagnosis of PD. In yet some further embodiment, the kits of the invention may be applicable for DP patients with motor symptoms, specifically, PD-M. In yet some further embodiments, the kits of the invention may be particularly applicable for use in monitoring and assessing responsiveness of a mammalian subject suffering from at least one synucleinopathy to a treatment regimen. More specifically, the kits of the invention may be useful for any of the methods as described herein. Still further, in some embodiments, the kits of the invention may be used for assessing if a subject diagnosed with PD is likely to develop dementia. More specifically, these specific kits may be adapted for use in the methods for assessing and predicting if a subject diagnosed with PD is likely to develop dementia, in accordance with the invention. In yet some further embodiments, the kits of the invention may be used for methods for the assay of PSer129 α-Syn in a biological sample. In further embodiments, kits for measuring the concentration of synucleins in a sample, are provided by the invention. More specifically, the kits may be used for the diagnosis of synucleinopathy-related diseases and/or cancer, and or for the monitoring of disease progression or the effect of a treatment regime. The kits comprise immobilized lipids to which the synucleins are capable of binding, and least one anti-PSer129 synuclein antibody or aptamer. Generally, the kit may also comprise a second antibody labeled with a visible tag, for example Horseradish peroxidase (HRP) or Chemiluminescence tag. Still further, as explained herein above, a key inventive feature of the present invention is the fact that the ELISA assay disclosed and claimed herein is based on the measurement of P-Ser129 α-Syn whilst bound to membrane lipids and separated from the non-bound components of the sample. These proteins essentially lack secondary and tertiary structure when present in their free cytosolic, unbound form, and only adopt their functional, folded forms upon binding to lipids, or any other hydrophobic agent. Without wishing to be bound by theory, the use of membrane lipids in the presently-disclosed ELISA to bind the PSer129 α-Syn that are present in the sample being tested, leads to at least the following two novel and unexpected advantages of the present invention:

First, the form of the synuclein being assayed (PSer129 α-Syn) is the functionally-relevant (and hence pathogenically-relevant) form of the protein which by one option is lipid bound and by another option is both lipid bound and proteinase K-resistant form or lipid bound and P-Ser129 α-Syn bound.

Second, epitope recognition by the antibodies used in the Lipid-ELISA is enhanced following folding of the proteins (possibly by creation of new three-dimensional binding sites), thereby leading to greatly-enhanced sensitivity, and reduced minimal threshold detection values.

A further aspect of the invention relates to a method for the assay of PSer129 α-Syn in a whole blood sample. More specifically, the method of the invention may comprise the steps of:

First in step (a), contacting said whole blood sample or at least one aliquot thereof with immobilized lipids under conditions enabling binding of the synucleins to the lipids.

In the next step (b), detecting the lipid-bound PSer129 α-Syn by at least one agent that specifically recognizes and binds said PSer129 α-Syn.

The term “assay” is used herein to refer to both detection (i.e. the determination of the presence or absence of PSer129 α-Syn in the sample tested in a binary yes/no manner) as well as to the quantitative determination of the concentration of the relevant PSer129 synuclein in the sample.

Still further, when a quantitative determination is being performed, the results obtained from the assay of the present invention is compared with results obtained with standardized amounts of pure PSer129 α-Syn. Thus, in some embodiments, the invention provides methods for determining the presence and/or amount of post translationally modified α-synuclein, specifically, PSer129 α-Syn in a sample. The method comprising contacting the sample or at least one aliquot thereof or any protein preparation thereof with synuclein-binding lipids immobilized to a solid support, under conditions that allow binding of the synuclein in the sample to said immobilized lipids, thereby forming an immobilized complex of said synuclein and lipids. The unbound synuclein is eliminating by washing away unbound molecules.

In yet some further embodiments, the methods of the invention may be also applicable for assaying other post-translationally modified α-Syn, for example, α-Syn that is modified in at least one amino acid residue, by at least one of phosphorylation, nitration, sumoylation, acetylation and glycation.

In yet some further embodiments, the methods of the invention may be applicable for assaying phosphorylate α-Syn, specifically, α-Syn that is modified by a phosphorylation in at least one of Serine 129, Serine 87, Tyrosine 125, Tyrosine 133 and Tyrosine 136 of said α-Syn. In yet some further embodiments, the methods of the invention may be applicable for determining α-Syn modified by a nitration is in at least one of Tyrosine 39, Tyrosine 125, Tyrosine 133 and Tyrosine 136 of said α-Syn. It should be understood that in cases that other post-translationally modified α-Syn are assayed, agents (e.g., antibodies and/or aptamers) that specifically recognize and bind such post-translationally modified α-Syn, are used by the methods of the invention.

In yet some further embodiments, the methods of the invention are particularly applicable for biological sample that is whole blood sample. Specifically, whole blood samples that comprise blood cells, specifically, erythrocytes and platelets.

In yet some further embodiments, such blood sample may be a hemoglobin depleted blood sample. In yet some further embodiments, the agent that specifically recognizes and binds the PSer129 α-Syn (or any of the optional post-translationally modified forms of α-Syn), is at least one of an antibody or any antigen-binding fragment thereof, an aptamer and any combinations thereof. In yet some further embodiments, the immobilized lipids used for the methods of the invention are synuclein-binding lipids attached or connected directly or indirectly to a solid support, said lipids comprise at least one of naturally occurring, purified or synthetic phospholipid/s, glycolipid/s, plasmalogen/s, sphingolipid/s, triglycerides, cholesterol, glycolipid/s, free fatty acids, eicosanoids, lipoproteins or proteolipids and any combinations thereof. In more specific embodiments, the lipids comprise at least one of phosphatidylinositol (PI), phosphatidylserine (PS), phosphatidylethanolamine (PE), GM-1, GM-2, GM-3, GM-4 gangliosides, phosphatidic acid, phosphatidylcholine, phosphatidylserine, phosphatidylglycerol, phosphatidylinositol, phosphoinositides (PIPn), cardiolipin, ceramide, sphingomyelin, ether-phospholipids, plasmalogens, glycolipids, glucosylcerebroside, galactosylceramide lactosylceramide, cholesterol, cholesterol-ester, triglycerides, diglycerides, monoglycerides, fatty acids, lipoproteins or proteolipids and any combinations thereof.

In more specific embodiments, the immobilized lipids used by the methods of the invention comprise at least two of naturally occurring, purified or synthetic PI, PS, PE and GM-1 ganglioside. In yet some further embodiments, the lipids used by the methods of the invention are dissolved in at least one organic solvent prior to attachment to said solid support. Still further, in some embodiments, the organic solvent is at least one of ethanol, methanol, cyclohexene, chloroform, ether, isopropanol, and any combinations thereof. In some alternative embodiments, the methods of the invention may comprise an additional step of determining in at least one aliquot of said sample at least one of:

(a) total α-Syn amount; (b) amount of proteinase K-resistant α-Syn; (c) iron level;
(d) amount of oxidized α-Syn; (e) amount of S-nitrosylated α-Syn; (f) amount of heat-resistant α-Syn; (g) hemoglobin level; and (h) H-ferritin level.

All definitions, as defined and used herein, should be understood to control over dictionary definitions, definitions in documents incorporated by reference, and/or ordinary meanings of the defined terms.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to ±10%.

The indefinite articles “a” and “an,” as used herein in the specification and in the claims, unless clearly indicated to the contrary, should be understood to mean “at least one.” It must be noted that, as used in this specification and the appended claims, the singular forms “a”, “an” and “the” include plural referents unless the content clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

Throughout this specification and the Examples and claims which follow, all transitional phrases such as “comprising,” “including,” “carrying,” “having,” “containing,” “involving,” “holding,” “composed of,” and the like are to be understood to be open-ended, i.e., to mean including but not limited to. Specifically, it should understood to imply the inclusion of a stated integer or step or group of integers or steps but not the exclusion of any other integer or step or group of integers or steps. Only the transitional phrases “consisting of” and “consisting essentially of” shall be closed or semi-closed transitional phrases, respectively, as set forth in the United States Patent Office Manual of Patent Examining Procedures. More specifically, the terms “comprises”, “comprising”, “includes”, “including”, “having” and their conjugates mean “including but not limited to”. The term “consisting of” means “including and limited to”. The term “consisting essentially of means that the composition, method or structure may include additional ingredients, steps and/or parts, but only if the additional ingredients, steps and/or parts do not materially alter the basic and novel characteristics of the claimed composition, method or structure.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated herein above and as claimed in the claims section below find experimental support in the following examples. Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

### Experimental Procedures

### Samples and Preparation

BioFIND study samples (Kang U J, et al. (2016) Mov Disord. 2016; 31(6):924-32) of whole blood cell-pellets arrived frozen in dry ice. Samples were divided in aliquots and kept frozen at −80° C. until used. Each aliquot was thawed only once. Blood cell pellets were lysed in 1:5 volumes of cold double distilled water (DDW). A supernatant obtained following a spin at 17,000×g at 4° C. was used for measurements within one day from thawing. Samples were shuffled and randomly assigned for analysis. Each microtitter plate contained an equal number of HC and PD samples. All measurements were performed blinded to group identity.

Hemoglobin: Hemoglobin measurements were performed using the Triton X-100/NaOH method (Wolf H U, et al. (1984) Clin Chim Acta; 136(1):95-104), in which hemoglobin is converted to a colorimetric product that is determined at 574 nm. The concentration of hemoglobin was calculated according to a standard curve consisting of 0-2 μg/μl Hemin (Sigma, Rehovot, Israel).

H-ferritin: H ferritin was determined by sandwich-ELISA, with a monoclonal antibody rH02 as described previously (Cozzi A, et al. (2004) Blood; 103(6):2377-83). The standard curve consisted of the recombinant homo-polymer of H ferritin. Of note, L-ferritin levels in blood cell pellets were below limit of detection.

Iron: Iron was determined as described previously (Asperti M, et al. (2016) PLoS One; 11(10):e0164183). Briefly, acid extracted samples added to a chromogen reagent (1 volume of 0.1% bathophenanthroline sulfate and 1% thioglycolic acid solution; 5 volumes of water; and 5 volumes of saturated sodium acetate). Absorbance determined at 535 nm in parallel with known amounts of iron as a standard curve.

### α-Syn Detection by Lipid-ELISA

A PolySorp, 96-wells ELISA plate (Thermo Scientific, Getter, Israel) was coated with phosphatidylinositol (PI), phosphatidylserine (PS), phosphatidylethanolamine (PE) and GM-1 ganglioside (Sigma, Rehovot, Israel) at 1:1:1:1 w/vol. Lipids were dissolved either in methanol or cyclohexene and applied in a final amount of 5 μg total lipids per well. Plates were incubated overnight at 4° C. to complete evaporation of the solvent (immobilization of lipids). Blocking with 100 μl/well of 1% BSA (fatty acid-free) in PBS for one hour at 37° C., followed by one wash with PBS. Protein levels were determined by the Bradford method (Bradford M M. (1976) Anal Biochem; 72:248-54). Test samples were applied to the wells at the indicated protein amounts, in triplicates and incubated for 3 hours at 37° C. to allow capture of proteins by the immobilized lipids. The wells were washed with 3% H₂O₂ in DDW for 10 minutes and then with 1% formalin in PBS (without MgCl₂ or CaCl₂). A primary anti α-Syn antibody was added to the wells, as indicated (below). Following incubation for one hour at 37° C., the wells were washed 3 times and either processed for detection (primary antibody conjugated to HRP) or incubated with a secondary antibody for one hour at 37° C. (Jackson Laboratories, ENCO, Israel) diluted 1:8000 in 1% BSA in PBS and washed three times as above. Detection reaction as specified for each tested α-Syn form. The amounts of α-Syn were determined for each plate and each α-Syn form according to the linear phase of a standard curve consisting of a recombinant α-Syn protein, performed in parallel to the test samples. Assay sensitivity and specificity were described previously [16].

Total α-Syn: Lysed samples were diluted to a final protein concentration of 0.8 μg/μl in 1% BSA free fatty acid in PBS (when immobilized lipids were dissolved in methanol) or in 0.1M sodium phosphate pH 8 (when immobilized lipids were dissolved in cyclohexene). Total α-Syn was determined using anti α-Syn ab MJF-1 (Abcam, Tel-Aviv, Israel)—HRP conjugated. Detection reaction with 100 μl of TMB one component microwell substrate (Southern Biotech, Birmingham, Ala., USA) per well. The reaction was terminated with 100 μl/well of 1M H₂SO₄. Absorbance at 450 nm was determined using a plate-reader (EL808 Ultra Microplate Reader, Bio-Tek Instruments, VT, USA). Standard curve with purified human wt α-Syn protein. Total α-Syn was determined at three different protein amounts 8, 16 and 32 μg/well, in triplicates (experiment 1) or at 16 μg of total proteins in triplicates (experiment 2 and 3)

PK^{res} α-Syn determined following incubation of 12 mg total protein with PK (0.6 μg/μl; #3115887001, Sigma, Rehovot, Israel) in homogenization buffer [20 mM HEPES pH 7.3; 1 mM MgCl₂, 0.32M sucrose; 43 mM β-ME]. Following 25 minutes of incubation at 37° C., samples were transferred to 90° C. for 10 minutes, to inactivate the protease and spun at 17000×g for 30 minutes. PK^{res} α-Syn levels were determined using anti α-Syn ab MJF-1 (Abcam, Tel-Aviv, Israel)—HRP conjugated and detection reaction with TMB as above. PK^{res} α-Syn was determined at 40, 80, 120 μg proteins (representing protein amounts before PK treatment), in 1% BSA in PBS, in triplicates (experiment 1) or at 80 μg proteins (before the PK treatment) per well in 1% BSA/PBS, in triplicates (experiment 2). The immobilized lipids were dissolved in methanol.

PSer129 α-Syn: detection following hemoglobin clearance with HemoVoid kit (Biotech Support Group LLC, NJ, US). Samples of 4 mg lysed blood cells in homogenization buffer containing 1 mM PMSF and 1 mM NaF were applied into a tube containing 50 mg HemoVoid matrix. The eluted fraction was diluted 1:2 in 0.1M sodium-phosphate pH 8 and applied at 0.16 mg (the equivalent amount of protein before HemoVoid clearance) per well on white PolySorp ELISA plates (Thermo Scientific) pre-coated with lipids dissolved in cyclohexene. Samples reacted with anti PSer129 α-Syn monoclonal antibody (pSyn#64, WAKO, Osaka, Japan). A standard curve consisting of recombinant protein, phosphorylated at Ser129 (Proteos, Kalamazoo, US), was applied in parallel to test samples and used as a reference. Super Signal ELISA Femto (Pierce, Ornat, Israel) was used for the enzymatic reaction. Luminescence was determined by Luminometer (Infinite M200 Pro. NEOTEL Scientific Instrumentation Ltd) immediately after adding the substrate.

### Oxydized α-Syn:

Samples treated with HemoVoid (see above for PSer129 α-Syn) and applied on microtitter plate pre-coated with lipids dissolved in cyclohexene. Detection using anti oxidized α-Syn ab, syn 303 (Biolegend, ENCO, Petach Tikvah, Israel). Detection reaction with TMB (as above). A standard curve consisting of a recombinant α-Syn protein that was oxidized by ferrous sulfate (0.5 mM) and H₂O₂ (1 mM) was applied in each plate as a reference.

### Statistical Analyses

The groups were compared by Kruskal Wallis test as implemented by the PMCMRplus package of R (Pohlet T. (2018) R package version 1.4.0) using Conover's post hoc test for the pairwise comparisons (Conover W J. (1999) 3rd ed. NY: Wiley).

Differentiations between groups were analyzed by logistic regression of variables, using SPSS software. α-Syn forms with P values ≤0.5 and variables with P values of up to 10% were selected and a constant parameter was calculated to each variable. According to the equation, a personalized score (Z) was calculated and used to yield a predicted probability of PD. A receiver operating characteristic (ROC) curve was calculated according to the predicted probabilities. All data were analyzed using a qualified statistical software package (SPSS for Windows, Version 25.0, SPSS Inc., Chicago, Ill., USA). A P-value of less than 0.05 was considered significant. Prism8 (Graphpad) was used for graphics and calculations of Pearson's correlations.

### Example 1

### Determining Concentrations of α-Syn Forms in Test Samples

Demographic features of 45 HC and 46 PD participants are presented in Table 1. Age, race and education similarly varied between the groups (Table 1). The PD group was subdivided according to presentation of symptoms to PD-motor (PD-M, and PD with cognitive impairment, represented by MoCA ≤25 (PD-D). The concentrations of total protein, hemoglobin, H-ferritin and iron varied within groups with no significant differences between groups (Table 2).

In addition, oxidized α-Syn levels, detected by the syn303 antibody, were determined in samples pre-treated with HemoVoid. Samples were applied on microtitter plates, pre-coated with lipids dissolved in cyclohexene. An amount of 0.022±0.012 μg oxidized α-Syn/mg protein was detected in the HC group. These levels did not differ from the levels detected in the PD-M or PD-D groups (Table 2).

TABLE 1
Demographic and clinical features in test groups

| | PD-M | PD-D | Healthy |
|---|---|---|---|
| | subjects | subjects | Control |
| Variable | (n = 32) | (n = 14) | (n = 45) |
| Age | | | |
| Mean (SD) | 70 (6) | 74.1 (7.6) | 69.5 (8.3) |
| (Min, Max) | (60, 83) | (59, 84) | (57, 96) |
| Gender | | | |
| Male | 20 (62%) | 11 (78%) | 19 (42%) |
| Female | 12 (38%) | 3 (22%) | 26 (58%) |
| Ethnicity | | | |
| Hispanic/Latino | 1 (3%) | 0 (0%) | 3 (6%) |
| Not Hispanic/ | 31 (97%) | 14 (100%) | 42 (94%) |
| Latino | | | |
| Race | | | |
| White | 32 (100%) | 14 (100%) | 38 (87.5%) |
| | | (95%) | |
| African-American | 0 (0%) | 0 (0%) | 5 (11%) |
| Asian | 0 (0%) | 0 (0%) | 1 (2%) |
| Other | 0 (0%) | 0 (5%) | 1 (2%) |
| Education | | | |
| <13 years | 2 (6%) | 2 (14%) | 3 (7%) |
| 13-23 years | 30 (94%) | 12 (86%) | 41 (91%) |
| >23 years | 0 (0%) | 0 (0%) | 1 (2%) |
| Family history | | | |
| positive | 13 (40%) | 2 (14%) | 5 (11%) |
| negative | 19 (60) | 12 (86%) | 40 (89%) |
| MDS-UPDRS | | | |
| MDS-UDPRS total | 53.5 (19.5) | 59.3 (31.7) | N/A |
| MDS-UDPRS I | 3.03 (2.5) | 4.3 (2.5) | N/A |
| MDS-UDPRS II | 10.56 (5.7) | 12 (5.7) | N/A |
| MDS-UDPRS III | 37.6 (14) | 38.3 (14) | N/A |
| MDS-UDPRS IV | 2.5 (2.6) | 4.6 (2.6) | N/A |
| H&Y | | | |
| Stage 1-2 | 18 (56%) | 11 (79%) | N/A |
| Stage 3 | 10 (31%) | 2 (14%) | N/A |
| Stage 4 | 4 (13%) | 1 (7%) | N/A |
| Stage 5 | 0 (0%) | 0 (0%) | N/A |
| MoCA | | | |
| Mean (SD) | 28.1 (1.3) | 23 (2.1) | 27.1 (1.4) |
| (Min, Max) | (26, 30) | (18, 25) | (26, 30) |
| REM | | | |
| Negative (<5) | 20 (63%) | 7 (50%) | 38 (84%) |
| Positive (≥5) | 12 (27%) | 7 (50%) | 7 (26%) |

PD-M, Parkinson's disease with motor symptoms; PD-D, Parkinson's disease with cognitive impairment; Family history, at least one affected family member (positive), or no affected family member (negative); MDS-UPDRS, Movement Disorders Society-Unified Parkinson's Disease Rating Scale Motor score, mean (SD); H&Y, Hoehn and Yahr; MoCA, Montreal Cognitive Assessment; RBDSQ, REM Sleep Behavior Disorder Screening Questionnaire.

TABLE 2
Concentrations determined in test samples

| α-Syn | PD-M | PD-D | HC | P (PD-M | P (PD-M |
|---|---|---|---|---|---|
| form^{a} | (n = 32) | (n = 14) | (n = 45) | vs HC) | vs PD-D) |
| Total | | | | | |
| (methanol) α- | | | | | |
| Syn^{b} | | | | | |
| Average | 0.307 ± 0.10 | 0.241 ± 0.05 | 0.261 ± 0.06 | 0.02 | 0.01 |
| Experiment 1 | 0.304 ± 0.12 | 0.239 ± 0.05 | 0.249 ± 0.09 | 0.04 | *0.06 |
| Experiment 2 | 0.317 ± 0.09 | 0.241 ± 0.06 | 0.273 ± 0.07 | *0.06 | 0.01 |
| Total | 0.56 ± 0.11 | 0.58 ± 0.13 | 0.55 ± 0.12 | ns | ns |
| (cyclohexene) | | | | | |
| α-Syn^{b} | | | | | |
| PK^{res} | | | | | |
| α-Syn^{b} | | | | | |
| Average | 0.044 ± 0.03 | 0.024 ± 0.01 | 0.030 ± 0.02 | 0.01 | 0.01 |
| Experiment 1 | 0.048 ± 0.035 | 0.028 ± 0.023 | 0.032 ± 0.027 | 0.01 | 0.04 |
| Experiment 2 | 0.0409 ± 0.036 | 0.020 ± 0.013 | 0.028 ± 0.024 | 0.04 | 0.03 |

| | | | | | *P (PD- |
|---|---|---|---|---|---|
| α-Syn | PD-M | PD-D | HC | *P (PD-M | M vs PD- |
| form^{c} | (n = 32) | (n = 14) | (n = 45) | vs HC) | D) |
| PSer129^{d} | 35.82 ± 15.19 | 27.37 ± 9.76 | 24.48 ± 7.6 | 0.001 | *0.07 |
| Syn 303 ^{b} | 0.024 ± 0.09 | 0.021 ± 0.07 | 0.022 ± 0.012 | ns | ns |

| Blood | PDM | PD-D | HC | *P (PD-M | *P (PD-M |
|---|---|---|---|---|---|
| parameters | (n = 42) | (n = 17) | (n = 64) | vs HC) | vs PD-D) |
| Protein^{e} | 80.08 ± 15.3 | 93.81 ± 14.3 | 87.89 ± 17.5 | ns | ns |
| Hemoglobin^{f} | 0.127 ± 0.01 | 0.113 ± 0.01 | 0.125 ± 0.01 | ns | ns |
| Iron⁸ | 0.274 ± 0.06 | 0.26 ± 0.07 | 0.278 ± 0.07 | ns | ns |
| H-ferritin^{h} | 13.64 ± 7.9 | 16.12 ± 16.7 | 14.56 ± 8.2 | ns | ns |

^{a}mean ± sd;
^{b}μg/mg protein;
^{c}hemoglobin clearance with HemoVoid;
^{d}pg/mg protein;
^{e}mg/ml lysed blood cell pellets;
^{f}mg/mg protein;
^{g}ng/mg protein;
^{h}ng/mg protein.

Protein levels determined per volume of lysed blood cell pellet samples. The concentrations of all other variables presented per mg total proteins (mean±SD). P value calculated by Kruskal Wallis and Conover's post hoc test for the pairwise comparisons; * tend to be significant; ns, not significant.

### Example 2

Higher Levels of Total α-Syn in PD-M than HC Samples

Total α-Syn levels were determined in experiments 1 and 2 using methanol for lipid immobilization in the assay. Closely similar total α-Syn levels were detected in both measurements (Table 2; r=0.5; P value of correlation <0.0001), showing repeatability of the method. The average amount of total α-Syn (in experiments 1 and 2) determined in the HC samples, 0.261±0.06 μg α-Syn/mg protein, was somewhat lower (P=0.07, Kruskal Wallis) than the amounts determined in the entire PD group (0.286±0.09 μg α-Syn/mg protein). However, excluding PD-D samples, defined by MoCA 25, resulted in a significant difference between the PD-M (0.307±0.10 μg α-Syn/mg protein) and HC group (P=0.02, Kruskal Wallis; FIG. 1A). Total α-Syn levels in the PD-M group correlated with disease severity, represented by UPDRS, with r=0.2 and P=0.02 (FIG. 1B). Interestingly, total α-Syn levels differed between the two PD sub-groups. The levels determined in the PD-D group (0.241±0.05 μg α-Syn/mg protein) were significantly lower than the PD-M group (P=0.01 Kruskal Wallis; FIG. 1A and Table 2).

Total α-Syn levels determined in HC samples using cyclohexene for lipid immobilization were 0.55±0.12 μg α-Syn/mg protein, which is −2 folds higher than α-Syn levels determined with methanol as a solvent for the lipids. However, no significant differences between the HC and PD groups were detected (Table 2). Thus, cyclohexene improved the sensitivity, resulting in detection of higher α-Syn levels, however, at the expense of the power of the assay to differentiate between the groups.

### Example 3

Higher Levels of Proteinase K-Resistant (PK^{res}) α-Syn in PD-M than HC Samples

PK^{res} α-Syn levels were determined in experiments 1 and 2 with closely similar values (Table 2; r=0.6; P value of correlation <0.0001). The average PK^{res} α-Syn levels in the HC group, determined in the two experiments (0.030±0.02 μg α-Syn/mg protein) is significantly lower than the amounts determined in the PD-M group (0.044±0.03 μg α-Syn/mg protein; P=0.01, Kruskal Wallis). In addition, PK^{res} α-Syn levels significantly differ between PD-M and PD-D (0.024±0.01 μg α-Syn/mg protein) groups (P=0.01, Kruskal Wallis; FIG. 1C and Table 2). PK^{res} α-Syn levels in the PD-M group correlate with disease severity, represented by UPDRS, with r=0.23 and P=0.02 (FIG. 1D).

### Example 4

Higher Levels of Phospho Serine (PSer) 129 α-Syn in PD-M than HC Samples

PSer129 α-Syn was shown to bind membrane lipids [18] and therefore can be determined by the Lipid-ELISA assay. PSer129 α-Syn levels in blood cells are considerably lower than total α-Syn and occur at the picogram (pg) scale. To enable its detection, samples were treated to remove hemoglobin using HemoVoid and detection was performed using cyclohexene for lipid immobilization. PSer129 α-Syn levels determined in the HC group were 24.48±7.6 pg α-Syn/mg protein. These levels are significantly lower than the levels detected in the PD-M group, 35.82±15.19 pg α-Syn/mg protein (P value=0.001; Kruskal Wallis; FIG. 1E and Table 2). PSer129 α-Syn levels in the PD-M group correlate with disease severity, represented by UPDRS, with r=0.49 and P<0.0001 (Fig. F). Interestingly, PSer129 α-Syn levels determined in the PD-D group, 27.37±9.76 pg α-Syn/mg protein (n=14) showed a tendency for a difference with PD-M group (P=0.07, Kruskal Wallis).

### Example 5

### α-Syn Levels in Blood Cells Differentiates HC and PD Groups

The concentrations of total α-Syn (average of experiments 1 and 2), PK^{res} α-Syn (average of experiments 1 and 2), PSer129 α-Syn and iron were used to develop a diagnostic algorithm for differentiating PD-M and healthy controls. A specific Z-value that is calculated using the following equation:

Z=−2.412+(−18.229×iron)+(4.412×total MeOH)+(40.847×PKres)+(0.155×PSer129)

is used to calculate P(predict), a value used to determine the degree of discrimination between the test groups.

$$P ( predict ) = \frac{1}{1 + {e}^{- Z}}$$

Cut off was set at =0.5, where P(predict)<0.5 is HC and P(predict) >0.5 is PD.

The composite biomarker demonstrates a strong classification between the PD-M and HC groups (FIG. 2A); a strong correlation with UPDRS, with r=0.60 and P<0.0001 (FIG. 2B); and provides an AUC=0.85 with 0.69 sensitivity and 0.91 specificity (FIG. 2C)

In addition, the composite biomarker demonstrate an AUC=0.75 between PD subgroups, PD-M and PD-D. This degree of classification between the PD subgroups does not allow classification, yet, it provides an indication for a biochemical difference.

### Example 6

### Optimization of the PSer129 α-Syn Binding Assay

In the process of optimizing the assay for PSer129 α-Syn binding, the effect of different non-ionic detergents such as CHAPS, CHAPSO and Brij-35 was tested. Detection of purified α-synuclein or purified Pser129 α-synuclein (Michael J. Fox foundation, Proteos Ins.) was tested following wash steps with washing solutions comprising CHAPS. As shown in FIG. 3, it appears that including either one of these detergents in wash solutions, enhanced specific PSer129 α-Syn detection.

1. A method for the detection or diagnosis of at least one synucleinopathy in a subject, the method comprising the steps of:
(a) determining the amount of α-synuclein (α-Syn) phosphorylated at Serine 129 (PSer129 α-Syn) in at least one whole blood sample of said subject, or in at least one aliquot thereof; to obtain a PSer129 α-Syn value of the sample, wherein said whole blood sample comprises blood cells;
(b) determining if the value obtained in step (a) is any one of positive or negative with respect to a predetermined standard PSer129 α-Syn value or to a PSer129 α-Syn value in at least one control sample;
wherein a positive value of said PSer129 α-Syn in said sample, indicates that said subject suffers from at least one synucleinopathy.

2. The method according to claim 1, for the detection or diagnosis of at least one synucleinopathy in a subject, the method comprising the steps of:
(a) determining the amount of PSer129 α-Syn in at least one whole blood sample of said subject by:
(i) contacting said whole blood sample or at least one aliquot thereof with immobilized lipids and/or at least one hydrophobic agent, under conditions enabling binding of said synucleins to said lipid/s and/or hydrophobic agent/s; and
(ii) detecting the lipid-bound and/or hydrophobic agent-bound PSer129 α-Syn by at least one agent that specifically recognizes and binds said lipid-bound and/or hydrophobic agent-bound PSer129 α-Syn; to obtain a PSer129 α-Syn value of the sample; and
(b) determining if the value obtained in step (a) is any one of positive or negative with respect to a predetermined standard PSer129 α-Syn value or to a PSer129 α-Syn value in at least one control sample;
wherein a positive value of said PSer129 α-Syn in said sample, indicates that said subject suffers from at least one synucleinopathy;
optionally, at least one of: (i) said whole blood sample is a hemoglobin depleted sample; and (ii) said whole blood sample comprises blood cells, either intact or lysed.

3-4. (canceled)

5. The method according to claim 1, further comprising the steps of determining the value of at least one additional parameter in said sample or at least one aliquot thereof, said method comprising the steps of:
(a) determining the amount of PSer129 α-Syn in at least one whole blood sample of said subject, or in at least one aliquot thereof, to obtain a PSer129 α-Syn value of the sample;
(b) determining in at least one aliquot of said sample at least one of:
(i) total α-Syn amount, to obtain an α-Syn value of the sample;
(ii) amount of proteinase K (PK)-resistant α-Syn, to obtain a PK resistant α-Syn value of the sample;
(iii) iron level, to obtain an iron value of the sample;
(iv) amount of oxidized α-Syn, to obtain an oxidized α-Syn value of the sample;
(v) amount of S-nitrosylated α-Syn, to obtain an α-Syn value of the sample;
(vi) amount of heat-resistant α-Syn, to obtain an α-Syn value of the sample;
(vii) hemoglobin level to obtain an α-Syn value of the sample; and
(viii) H-ferritin level, to obtain an α-Syn value of the sample;
(c) calculating the weighed sum of said PSer129 α-Syn value as determined in step (a) and of at least one of the values as defined in step (b), to obtain a Sum value of the sample;
(d) determining if the Sum value obtained in step (c) is any one of positive or negative with respect to a predetermined standard Sum value or to a Sum value of at least one control sample;
wherein a positive Sum value indicates that said subject suffers from at least one synucleinopathy.

6. The method according to claim 5, comprising the steps of:
(a) determining the amount of PSer129 α-Syn in at least one whole blood sample of said subject, or in at least one aliquot thereof, to obtain a PSer129 α-Syn value of the sample;
(b) determining in at least one aliquot of said sample the total α-Syn amount, to obtain an α-Syn value of the sample;
(c) determining in at least one aliquot of said sample the amount of proteinase K-resistant α-Syn, to obtain a PK resistant α-Syn value of the sample;
(d) determining in at least one aliquot of said sample the iron level, to obtain an iron value of the sample;
(e) calculating the weighed sum of said PSer129 α-Syn value as determined in step (a), the α-Syn value as determined in step (b), the PK resistant α-Syn value as determined in step (c), and the iron value as determined in step (d), to obtain a Sum value;
(f) determining if the Sum value obtained in step (e) is any one of positive or negative respect to a predetermined standard Sum value or to a Sum value in at least one control sample;
wherein a positive Sum value calculated in said sample, indicates that said subject suffers from at least one synucleinopathy.

7. The method according to claim 5, wherein at least one of:
(i) a total α-Syn value of the sample is obtained by a method comprising:
(a) contacting said sample or at least one aliquot thereof with immobilized lipids and/or at least one hydrophobic agent under conditions enabling binding of the synucleins to the lipids and/or the hydrophobic agent/s; and
(b) detecting the lipid-bound and/or hydrophobic agent-bound α-Syn by at least one agent that specifically recognizes and binds said lipid-bound and/or hydrophobic agent-bound α-Syn, to obtain a total α-Syn value of the sample;
and
(ii) a PK resistant α-Syn value of the sample is obtained by a method comprising:
(a) contacting said sample or at least one aliquot thereof with proteinase K;
(b) contacting said proteinase K treated sample obtained in step (a) with immobilized lipids and/or at least one hydrophobic agent under conditions enabling binding of the synucleins to the lipids; and
(c) detecting the lipid-bound and/or hydrophobic agent-bound proteinase K resistant α-Syn by at least one agent that specifically recognizes and binds said lipid-bound and/or hydrophobic agent-bound α-Syn, to obtain a PK resistant α-Syn value of the sample.

8. (canceled)

9. The method according to claim 2, wherein said agent that specifically recognizes and binds said PSer129 α-Syn is at least one of an antibody or any antigen-binding fragment thereof, an aptamer and any combinations thereof.

10. The method according to claim 2, wherein said immobilized lipids are synuclein-binding lipids attached or connected directly or indirectly to a solid support, said lipids comprise at least one of naturally occurring, purified or synthetic phospholipid/s, glycolipids, plasmalogen/s, sphingolipid/s, triglycerides, cholesterol, steroids lipoproteins, proteolipids, free fatty acids, eicosanoids and any combinations thereof, optionally, said immobilized lipids comprise at least two of naturally occurring, purified or synthetic phosphatidylinositol (PI), phosphatidylserine (PS), phosphatidylethanolamine (PE) and GM-1 ganglioside.

11-12. (canceled)

13. The method according to claim 2, wherein said lipids are dissolved in at least one organic solvent prior to attachment to said solid support.

14. (canceled)

15. The method according to claim 1, wherein said synucleinopathy is at least one of Parkinson's disease (PD), Lewy body dementia (LBD) and multiple system atrophy (MSA), optionally, said PD, is PD with motor symptoms (PD-M).

16-17. (canceled)

18. The method according to claim 1, wherein said method further comprises the step of administering to a subject diagnosed with said at least one synucleinopathy, a therapeutically effective amount of a therapeutic agent for said synucleinopathy.

19. The method according to claim 1, for at least one of
(I) determining the severity and progression of said at least one synucleinopathy in a diagnosed subject;
(II) assessing and/or predicting if a subject diagnosed with PD is likely to develop dementia; and
(III) monitoring and assessing responsiveness of a mammalian subject suffering from at least one synucleinopathy to a treatment regimen, said method comprises the steps of:
(a) determining the amount of PSer129 α-Syn in at least one whole blood sample of said subject, or in at least one aliquot thereof; to obtain a PSer129 α-Syn value of the sample; and optionally determining at least one of: (i) the total α-Syn amount, to obtain an α-Syn value of the sample; (ii) the amount of proteinase K-resistant α-Syn, to obtain a PK resistant α-Syn value of the sample; and (iii) the iron level, to obtain an iron value of the sample;
(b) calculating the weighed sum of said PSer129 α-Syn value as determined in step (a), and optionally of at least one of the α-Syn value as determined in step (a i), the PK resistant α-Syn value as determined in step (a ii), and the iron value as determined in step (a iii), to obtain a Sum value of the sample;
(c) repeating steps (a) and (b) to obtain a Sum value for at least one more temporally-separated sample;
(d) calculating the rate of change of said Sum values between said temporally-separated samples to obtain a rate of change Sum value; and
(e) determining if the rate of change Sum value obtained in step (d) is positive or negative with respect to a predetermined standard rate of change Sum value or to the rate of change Sum value calculated in at least one control whole blood sample;
wherein a positive rate of change Sum value indicates that said subject responds to said therapeutic regimen.

20-21. (canceled)

22. A kit comprising:
(a) immobilized lipids and/or at least one immobilized hydrophobic agent;
and
(b) at least one agent that specifically recognizes and binds PSer129 α-Syn;
said kit optionally further comprising at least one of:
(c) at least one agent that specifically recognizes and binds α-Syn;
(d) Proteinase K;
(e) means for determining iron levels in a sample;
(f) pre-determined calibration curve providing standard values;
(g) at least one control sample;
(h) at least one means for depleting hemoglobin from a whole blood sample,
said whole blood sample comprises blood cells; and
(i) at least one organic solvent.

23-24. (canceled)

25. The kit according to claim 22, wherein at least one of:
(a) said agent that specifically recognizes and binds said PSer129 α-Syn is at least one of an antibody or any antigen-binding fragment thereof, an aptamer and any combinations thereof;
(b) said whole blood sample is a hemoglobin depleted sample; and
(c) said whole blood sample comprises blood cells, either intact or lysed

26. The kit according to claim 22, wherein said immobilized lipids are synuclein-binding lipids attached or connected directly or indirectly to a solid support, said lipids comprise at least one of naturally occurring, purified or synthetic phospholipid/s, glycolipid/s, plasmalogen/s, sphingolipid/s, triglycerides, cholesterol, steroids, glycolipid/s, lipoproteins, proteolipids, free fatty acids, eicosanoids and any combinations thereof, optionally, said immobilized lipids comprise at least two of naturally occurring, purified or synthetic PI, PS, PE and GM-1 ganglioside.

27-29. (canceled)

30. The kit according to claim 22, for the detection or diagnosis of at least one synucleinopathy in a subject, optionally, said synucleinopathy is PD, optionally, said PD is PD-M.

31-32. (canceled)

33. The kit according to claim 22, for at least one of:
(i) monitoring and/or assessing responsiveness of a mammalian subject suffering from at least one synucleinopathy to a treatment regimen;
(ii) assessing and/or predicting if a subject diagnosed with PD is likely to develop dementia; and
(iii) assaying PSer129 α-Syn in a whole blood sample.

34-35. (canceled)

36. A method for the assay of PSer129 α-Syn in a whole blood sample, wherein said whole blood sample comprises blood cells, the method comprising:
(a) contacting said whole blood sample or at least one aliquot thereof with immobilized lipids and/or at least one immobilized hydrophobic agent under conditions enabling binding of the synucleins to the lipids;
and
(b) detecting the lipid-bound and/or hydrophobic agent-bound PSer129 α-Syn by at least one agent that specifically recognizes and binds said PSer129 α-Syn.

37-38. (canceled)

39. The method according to claim 36, wherein at least one of:
(a) said agent that specifically recognizes and binds said PSer129 α-Syn is at least one of an antibody or any antigen-binding fragment thereof, an aptamer and any combinations thereof;
(b) said whole blood sample is a hemoglobin depleted sample; and
(c) said whole blood sample comprises blood cells, either intact or lysed.

40. The method according to claim 36, wherein said immobilized lipids are at least one of:
(i) synuclein-binding lipids attached or connected directly or indirectly to a solid support, said lipids comprise at least one of naturally occurring, purified or synthetic phospholipid/s, glycolipid/s plasmalogen/s, sphingolipid/s, triglycerides, cholesterol, glycolipid/s, free fatty acids, eicosanoids, lipoproteins or proteolipids and any combinations thereof, optionally, said immobilized lipids comprise at least two of naturally occurring, purified or synthetic PI, PS, PE and GM-1 ganglioside; and
(ii) said lipids are dissolved in at least one organic solvent prior to attachment to said solid support.

41-44. (canceled)

45. The method according to claim 36, wherein said method further comprises the step of determining in at least one aliquot of said sample at least one of:
(a) total α-Syn amount;
(b) amount of proteinase K-resistant α-Syn;
(c) iron level;
(d) amount of oxidized α-Syn;
(e) amount of S-nitrosylated α-Syn;
(f) amount of heat-resistant α-Syn;
(g) hemoglobin level; and
(h) H-ferritin level.
